(19) United States
(12) Patent Application Publication
(10) Pub. No.: US 2024/0008791 A1
(43) Pub. Date: Jan. 11, 2024

(54) METHOD OF PROCESSING ELECTROCARDIOGRAM SIGNAL

(71) Applicant: ATSENS CO., LTD., Gyeonggi-do (KR)

(72) Inventors: Kab Mun CHA, Seoul (KR); Jong Ook Jeong, Gyeonggi-do (KR)

(21) Appl. No.: 18/470,993

(22) Filed: Sep. 20, 2023

Related U.S. Application Data
(63) Continuation-in-part of application No. 18/345,751, filed on Jun. 30, 2023.
Application No. 18/345,751 is a continuation of application No. 17/177,726, filed on Feb. 17, 2021, now Pat. No. 11,730,416.

(30) Foreign Application Priority Data
Feb. 18, 2020 (KR) 10-2020-0019631
Apr. 21, 2023 (KR) 10-2023-0052825

(51) Int. Cl. A61B 5/352; A61B 5/0245; A61B 5/024; A61B 5/00; A61B 5/366; A61B 5/333

(52) U.S. Cl. CPC A61B 5/352; A61B 5/0245; A61B 5/02405; A61B 5/7267; A61B 5/366; A61B 5/0006; A61B 5/7203; A61B 5/7221; A61B 5/333

(57) ABSTRACT

A method includes steps of acquiring an electrocardiogram signal of an object, generating first signal segmentations by dividing the electrocardiogram signal according to a division rule, classifying the first signal segments into one or more preset groups using an electrocardiogram classification model, classifying second signal segments that do not belong to the one or more preset groups into an abnormal group, determining a noise section associated with the electrocardiogram signal among the second signal segments using a noise decision model, generating an analysis target section associated with the electrocardiogram signal excluding the noise section, and transmitting the analysis target section to an external device to analyze information related to a heart of the object.

### CROSS-REFERENCE TO RELATED APPLICATIONS

The present application claims priority to and is a continuation-in-part application of U.S. patent application Ser. No. 18/345,751, filed Jun. 30, 2023, which is a continuation of U.S. patent application Ser. No. 17/177,726 filed Feb. 17, 2021 (now U.S. Pat. No. 11,730,416 issued Aug. 22, 2023), which claims the benefit of priority under 35 U.S.C. § 119 to Korean Patent Application No. 10-2020-0019631, filed on Feb. 18, 2020, in the Korean Intellectual Property Office. The present application further claims priority under 35 U.S.C. § 119 to Korean Patent Application No. 10-2023-0052825, filed on Apr. 21, 2023, in the Korean Intellectual Property Office. All sections of the aforementioned application(s) and/or patent(s) are incorporated herein by reference in their entirety as if they are fully set forth herein.

### BACKGROUND

### 1. Field

The present disclosure relates to a method of processing an electrocardiogram signal.

### 2. Description of Related Art

Various algorithms to detect an R-peak of an electrocardiogram signal may be searched through Google (www.google.com).

However, the detected R-peak may not be accurate due to noise of an electrocardiogram signal. When an RR interval, which is an interval between two neighboring R-peaks, is calculated by using the R-peaks detected inaccurately as such, the RR interval may also be inaccurate.

In addition, when an R-peak of an electrocardiogram signal is directly detected and calculates an RR interval manually, the amount of work is limited and thus, it is necessary to automate the detection method.

Procedia Technology 4, 2012, pages 873 to 877 (R-peak detection algorithm for ECG using double difference and RR interval processing, hereinafter, referred to as a technology of the related art) discloses a method of detecting an R-peak of an electrocardiogram signal and a method of calculating an RR interval which is an interval between the R-peaks by using the detected R-peak.

In the related art, in order to remove an incorrect R-peak while automatically calculating the RR interval, R-peaks are compared by using an average RR interval between five consecutive R peaks, and when the RR interval is abnormal, a second R-peak is removed to calculate the RR interval, thereby calculating a correct RR interval.

However, in a case in which R-peaks are simply compared by using an average RR interval and the RR interval is calculated by removing a second R-peak when the RR interval is abnormal, there is a question as to whether or not only an abnormal R-peak is detected.

### SUMMARY

One or more aspects of the present disclosure includes a non-transitory computer-readable storage medium storing instruction that, when executed by a processor, causes the processor to perform operations. The operations includes (i) acquiring an electrocardiogram signal of an object, (ii) generating first signal segmentations by dividing the electrocardiogram signal according to a division rule, (iii) classifying the first signal segments into one or more preset groups using an electrocardiogram classification model, (iv) classifying second signal segments that do not belong to the one or more preset groups into an abnormal group, (v) determining a noise section associated with the electrocardiogram signal among the second signal segments using a noise decision model, (vi) generating an analysis target section associated with the electrocardiogram signal excluding the noise section, and (vii) transmitting the analysis target section to an external device to analyze information related to a heart of the object.

One or more aspects of the present disclosure includes a method of processing an electrocardiogram signal by an electrocardiogram signal processing device including at least one processor. The method includes steps of (i) acquiring an electrocardiogram signal of an object, (ii) generating first signal segmentations by dividing the electrocardiogram signal according to a division rule, (iii) classifying the first signal segments into one or more preset groups using an electrocardiogram classification model, (iv) classifying second signal segments that do not belong to the one or more preset groups into an abnormal group, (v) determining a noise section associated with the electrocardiogram signal among the second signal segments using a noise decision model, (vi) generating an analysis target section associated with the electrocardiogram signal excluding the noise section, and (vii) transmitting the analysis target section to an external device to analyze information related to a heart of the object.

In at least one variant, the electrocardiogram classification model is learned using signal segments of electrocardiogram signals measured in a plurality of objects as input using a deep learning method or a machine learning model to classify the first signal segments into the one or more preset groups and classifying the second signal segments into the abnormal group using a degree of similarity.

In another variant, the electrocardiogram classification model is learned using signal segments of electrocardiogram signals measured in a plurality of objects as input using a deep learning method or a machine learning model to classify the first signal segments into the one or more preset groups and classifying the second signal segments into the abnormal group using a degree of complexity.

In another variant, the division rule is to divide into signal segments based on time intervals or QRS time intervals of peaks of the electrocardiogram signal. The division rule is adjustable based on whether or not a noise section is detected in a previous time-of-window.

In another variant, the noise decision model is to determine signal segments continuously generated more than a reference number of times as the noise section among the second signal segments.

In another variant, a reference number is a value determined using a deep learning model or a machine learning model learned with a plurality of electrocardiogram signals of a plurality of objects as input. The deep learning model or machine learning model is learned with signal segments including a noise section and signal segments not including a noise section. The electrocardiogram classification model includes a plurality of models and is operated by selecting a model according to a measurement position.

The present disclosure provides a method of processing an electrocardiogram signal which is capable of accurately determining an abnormal electrocardiogram signal by determining whether or not the electrocardiogram signal is abnormal through two steps, as a disclosure for solving the technical problems described above.

Additional aspects will be set forth in part in the description which follows and, in part, will be apparent from the description, or may be learned by practice of the presented embodiments of the disclosure.

A processing method using an electrocardiogram signal according to the present disclosure includes (a) selecting a candidate for an abnormal R-peak from the electrocardiogram signal, (b) determining an abnormal R-peak from among the candidates selected in (a), and (c) excluding the abnormal R-peak determined in (b) among all the R-peaks from the electrocardiogram signal.

Specifically, (a) described above may use at least one of an interval between the R-peaks of the electrocardiogram signal, and complexity of the electrocardiogram signal. When the interval between the R-peaks of the electrocardiogram signal is used, (a) described above includes (a-1-1) calculating the interval between the R-peaks of the electrocardiogram signal, and (a-1-2) selecting a corresponding R-peak as a candidate for an abnormal R-peak when the interval between the R-peaks calculated in (a-1-1) is less than a preset value. In addition, when using complexity of an electrocardiogram signal, (a) described above may include (a-2-1) calculating the complexity of a signal to a preset window size, and (a-2-2) selecting an R-peak included in a window in which the complexity of the signal calculated in (a-2-1) is out of a preset range as a candidate for an abnormal R-peak.

In addition, (b) described above desirably determine an abnormal R-peak from among candidates selected in (a) by comparing a waveform of an electrocardiogram signal during a preset time period based on a time representing the R-peak selected in (a) with a waveform of an electrocardiogram signal representing a normal R-peak. Here, the waveform of the electrocardiogram signal representing the normal R-peak is calculated by an average value of waveforms of the electrocardiogram signal for multiple R-peaks during a preset time period.

According to embodiments, a method of preferentially classifying a noise section of an electrocardiogram signal includes acquiring an electrocardiogram signal by an electrocardiogram signal processing device, setting, by the electrocardiogram signal processing device, signal segments obtained by segmenting the electrocardiogram signal as a total set, classifying, by the electrocardiogram signal processing device, the signal segments included in the total set as one or more preset groups, classifying, by the electrocardiogram signal processing device, one or more signal segments that do not belong to the one or more groups as an abnormal group, classifying, by the electrocardiogram signal processing device, temporally continuous signal segments with respect to the signal segments belonging to the abnormal group as a noise section, and setting, by the electrocardiogram signal processing device, signal segments of the electrocardiogram signal that are not classified as the noise section as an analysis target section.

In classifying the signal segments as one or more preset the groups, signal segments with high frequency among the signal segments may be set as one or more reference signal segments based on similarity of signal patterns and are classified into groups based on the reference signal segments.

In classifying the signal segments as one or more preset the groups, complexity values of the signal segments may be calculated, and the signal segments may be classified into one or more groups by using the complexity values of the signal segments with high frequency based on the complexity values.

In classifying the signal segments as one or more preset the groups, a first reference signal segment having a highest frequency may be selected based on similarity, the first reference signal segment may be compared with the signal segments included in the total set, and signal segments having similarity that is greater than or equal to preset similarity reference value may be classified as a group of the first reference signal segment.

In setting the signal segments as the total set, signal segments segmented based on patterns of peaks generated from the electrocardiogram signal may be set as the total set.

In setting the signal segments as the total set, signal segments segmented based on QRS time intervals of the electrocardiogram signal may be set as the total set.

In setting the signal segments as the total set, the electrocardiogram signal may be normalized before the electrocardiogram signal is segmented.

The noise section may include a noise signal generated due to a motion of a target object.

In acquiring the electrocardiogram signal, an electrocardiogram signal measured by using an electrocardiogram patch attached to one of body parts of a target object may be acquired, and the electrocardiogram signal may be measured in a different pattern according to an attached position.

According to embodiments, an electrocardiogram signal processing device includes a signal processor configured to receive an electrocardiogram signal, set signal segments obtained by segmenting the electrocardiogram signal as a total set, classify the signal segments included in the total set as one or more preset groups, classify one or more signal segments that do not belong to the one or more groups as an abnormal group, classify temporally continuous signal segments with respect to the signal segments belonging to the abnormal group as a noise section, and set signal segments of the electrocardiogram signal that are not classified as the noise section as an analysis target section, and a communication unit configured to receive the electrocardiogram signal and transmit the electrocardiogram signal of the analysis target section to an external device.

According to embodiments, an electrocardiogram signal processing device includes a signal processor, and a memory that stores executable instructions that, when executed by the signal processor, facilitate performance of operations. The operations includes arranging signal segments obtained by segmenting an electrocardiogram signal as a total set, classifying the signal segments included in the total set as one or more preset groups or an abnormal group, classifying temporally continuous signal segments with respect to the signal segments belonging to the abnormal group as a noise section, and setting signal segments of the electrocardiogram signal that are not classified as the noise section as an analysis target section, and a communication unit configured to receive the electrocardiogram signal and transmit the electrocardiogram signal of the analysis target section to an external device. The abnormal group includes signal segments outside a normal range.

In at least one variant, the operations further include setting signal segments with high frequency based on similarity of signal patterns among the signal segments as one or more reference signal segments and classifying the signal segments into groups based on the reference signal segments.

In another variant, the operations further include calculating complexity values of the signal segments and classifying the signal segments into one or more groups by using the complexity values of the signal segments with high frequency based on the complexity values.

In another variant, the operations further comprise selecting a first reference signal segment having a highest frequency based on similarity, comparing the first reference signal segment with the signal segments included in the total set, and classifying signal segments having similarity that is greater than or equal to preset similarity reference value as a group of the first reference signal segment.

In another variant, the operations further comprise setting signal segments segmented based on patterns of peaks generated from the electrocardiogram signal as the total set.

In another variant, the operations further comprise setting signal segments segmented based on QRS time intervals of the electrocardiogram signal as the total set. In another variant, the operations further include acquiring an electrocardiogram signal measured by using an electrocardiogram patch attached to one of body parts of a target object, and measuring in a different pattern according to an attached position.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a flowchart illustrating a method of processing an electrocardiogram signal, according to an embodiment of the present disclosure;

FIG. 2A is a graph provided by extracting an R-peak from an electrocardiogram signal;

FIG. 2B is an exemplary diagram of an abnormal R-peak in an electrocardiogram signal;

FIG. 3A is an exemplary diagram of a detection region in an electrocardiogram signal;

FIG. 3B is an exemplary diagram of an abnormal peak in a detection region in an electrocardiogram signal;

FIG. 4A is an exemplary diagram of windows of an electrocardiogram signal according to embodiments of the present disclosure;

FIG. 4B is an exemplary diagram of a window including an abnormal peak extracted according to embodiments of the present disclosure;

FIG. 4C is an exemplary diagram of complexity values calculated for windows of an electrocardiogram signal;

FIG. 5A is an exemplary diagram of R-peaks of an electrocardiogram signal generated according to embodiments of the present disclosure;

FIG. 5B is an exemplary diagram of signal waveforms of sections selected from an electrocardiogram signal;

FIG. 6A is an exemplary diagram of normal peaks extracted from an electrocardiogram signal;

FIG. 6B is an exemplary diagram of peak interval values of normal peaks;

FIG. 6C is an exemplary diagram of signal waveforms for each section;

FIG. 7A is an exemplary diagram of signal waveforms for each section and an average normal QRS pattern extracted from signal waveforms;

FIG. 7B is an exemplary diagram of whether each signal waveform is abnormal using an Average Normal QRS pattern;

FIG. 8A is a diagram illustrating a process of deleting abnormal peaks from an electrocardiogram signal;

FIG. 8B is a diagram illustrating a process of deleting abnormal peaks from an electrocardiogram signal;

FIG. 8C is an exemplary diagram of peak values extracted from the electrocardiogram signal;

FIG. 8D is an exemplary diagram of peak values excluding abnormal peaks from the ECG signal;

FIG. 9 is a flowchart that shows another viewpoint of FIG. 1;

FIG. 10 is one exemplary implementation view of an electrocardiogram measurement device according to embodiments;

FIG. 11A is a block diagram of an electrocardiogram measurement device according to embodiments;

FIG. 11B is a block diagram of an electrocardiogram signal processing device according to embodiments;

FIG. 12 illustrates a network environment including a user terminal and an electrocardiogram measurement device according to embodiments;

FIG. 13 illustrates a network environment including an electrocardiogram signal processing device according to embodiments, a user terminal, an electrocardiogram management server, and a computing device;

FIG. 14 is a flowchart illustrating a method of processing an electrocardiogram signal, according to embodiments;

FIG. 15 is a flowchart illustrating a method of setting, by an electrocardiogram signal processing device, a noise section by using a reference segment, according to embodiments;

FIG. 16 is a flowchart illustrating a method of setting, by an electrocardiogram signal processing device, a noise section by using a reference value, according to embodiments;

FIG. 17 is a flowchart illustrating a method of processing signal segments of an analysis target section, according to embodiments;

FIG. 18 is a flowchart illustrating another method of processing an electrocardiogram signal, according to embodiments;

FIG. 19 is an example diagram illustrating that an electrocardiogram signal is divided into signal segments, according to embodiments;

FIG. 20 is an example diagram illustrating a process of classifying electrocardiogram signals into respective groups, according to embodiments;

FIG. 21A is an example diagram illustrating signal segments belonging to a first group classified according to embodiments;

FIG. 21B is an example diagram illustrating signal segments belonging to a second group classified according to embodiments;

FIG. 21C is an example diagram illustrating signal segments belonging to a third group classified according to embodiments;

FIG. 21D is an example diagram illustrating signal segments belonging to a fourth group classified according to embodiments;

FIG. 21E is an example diagram illustrating signal segments belonging to a fifth group classified according to embodiments;

FIG. 21F is an example diagram illustrating signal segments belonging to an et cetera (etc.) group classified according to embodiments;

FIG. 22A is an example diagram of a section other than a noise section processed according to embodiments; and

FIG. 22B illustrates an example of a noise section processed according to embodiments.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, configurations and operations of the present disclosure will be described in detail with reference to embodiments of the present disclosure illustrated in the accompanying drawings.

The present disclosure may be variously changed and have various embodiments, and thus, various embodiments will be illustrated in the drawings and described in detail in the detailed description. Effects and characteristics of the present disclosure, and a method of achieving the effects and characteristics will be apparent with reference to the embodiments to be described below in detail together with the drawings. However, the present disclosure is not limited to the embodiments to be disclosed below and may be implemented in various forms.

Hereinafter, embodiments of the present disclosure will be described in detail with reference to the accompanying drawings, and when describing with reference to the drawings, the same or corresponding components are denoted by the same reference numerals, and redundant descriptions thereof will not be repeated.

In this specification, terms such as “learning” are not intended to refer to mental actions such as human educational activities but are intended to refer to performing machine learning through computational procedures.

In the following embodiments, terms such as “first” and “second” are not used in a limiting meaning but for the purpose of distinguishing one component from another component.

In the following examples, singular expressions include plural expressions unless the context clearly indicates otherwise.

In the following embodiments, a term such as “include” or “have” means that there are characteristics or components described in the specification, and do not preclude a possibility of adding one or more other characteristics or components.

In the drawings, components may be exaggerated or reduced in size for the sake of convenient description. For example, the size and thickness of each component illustrated in the drawings are randomly illustrated for the sake of convenient description, and thus the present disclosure is not limited to the illustration.

When a certain embodiment is implemented differently, a certain process order may also be performed differently from the described order. For example, two processes described in succession may also be performed substantially simultaneously or may be performed in an order opposite to the described order.

A method of processing an electrocardiogram signal according to the present disclosure may be implemented in the form of a computer program to be processed by a processor of a computing device.

Electrocardiogram is a graph in which a potential change in body surface according to mechanical activity of a heartbeat, such as myocardial contraction and expansion, is recorded, and meaning of “detecting the electrocardiogram” is the same as meaning of “detecting an electric potential” generated on the body surface according to the heartbeat of a target object.

The electrocardiogram may include repetition of a P-QRS-T section. P waveforms represent that a current signal is generated from the sinus node, which polarizes the atrium and contracts the myocardium of valve, and as the atrium depolarizes, the myocardium of valve may be relaxed again. When the atrioventricular node is in pace making, P waveform may be inverted and detected. The width of a P wave may indicate atrial conduction time.

P-Q is a resting period in which a current signal does not stimulate the heart and may be displayed as a straight line in the electrocardiogram signal. This section refers to a section in which the atrium polarized in response to P contracts.

A QRS complex refers to a section in which the ventricles are immediately polarized (R) and immediately unpolarized (S) when the atrioventricular node releases a current signal (Q). In this section, the myocardium of the ventricles of valves may contract.

S-T is between S and T and may be displayed in a straight line because a current signal does not stimulate the heart.

In the embodiment of the present disclosure, an electrocardiogram signal includes an electrocardiogram measured over time and may be a discrete signal or a continuous signal.

In the embodiment of the present disclosure, the measured electrocardiogram signal may is measured by 1-channel electrocardiogram measurement device and is changed depending on an attachment position or direction. An electrocardiogram signal measured from an object may also change as long as a measurement tool is not attached to the same position and/or in the same direction.

In the embodiment of the present disclosure, the degree of similarity and/or similarity indicates the degree of similarity between signal segments and may be a correlation coefficient quantifying the degree of linear correlation, measurement of similarity by comparing a QRS complex that is a waveform pattern, a value measuring similarity based on a preset value such as heart rate variability, a cosine value, and so on. Additionally, the degree of similarity and/or similarity may be calculated through clustering between patterns of signals.

In the embodiment of the present disclosure, a complexity value indicates the degree of complexity of an electrocardiogram signal and may be measured by using a mathematical technique, such as entropy measurement or multiscale analysis. The complexity value may be calculated based on irregularity or regularity of a signal. A signal without regularity may be calculated with high complexity. The complexity value may be calculated in a time domain or a frequency domain.

An electrocardiogram signal processing device according to embodiments of the present disclosure may detect a noise section in a measured electrocardiogram signal. The electrocardiogram signal processing device may group or cluster electrocardiogram signals to classify signal segments of the electrocardiogram signals into one or more groups and may set the signal segments not classified into groups to noise sections. A signal segment satisfying a preset criterion among the signal segments not classified into groups may be set to a noise section. The signal segments that do not belong to the noise section may be classified as an analysis target section.

Grouping or clustering signal segments of the electrocardiogram signals may be based on patterns of the signal segments. The measured electrocardiogram signal may include repetition of similar patterns of signal segments. A section deviating from similar patterns may correspond to noise. According to embodiments of the present disclosure, the electrocardiogram signal processing device may analyze a pattern of reference signal segments of the electrocardiogram signal and classify a section deviating a similar pattern as a noise section. According to embodiments of the present disclosure, the section deviating from a similar pattern may be determined as one or more reference signal segments or one or more reference values, such as complexity values. According to embodiments, signal segments may be classified through a normalization process of converting the signal segments into relative values and relative time standards.

A noise section of an electrocardiogram signal may be a section including noise in the electrocardiogram signal, and refers to a section including a signal that is not normally measured. Among the electrocardiogram signals, a section including a signal that is not normally measured may be an unnecessary section for analyzing information related to the heart of the object.

When an electrocardiogram signal can be measured for long time, for example 48 hours or 14 days, the electrocardiogram signal is recorded as a large amount of data, and the electrocardiogram signal may include a large amount of noise. Noise included in the electrocardiogram signal may be generated by movement of the object, for example, an object may perform a behavior, such as coughing, exercising, sitting, jumping, running, walking, sleeping, or lying. Such noise corresponds to a section that does not need to be analyzed. The time to analyze the electrocardiogram signal may be shortened if analysis is performed after removing unnecessary sections such as noise from the large-capacity electrocardiogram signal.

According to the known method, time-of-windows with a preset interval are set, and sections of the electrocardiogram signal may be classified into sections related to normal beat (N) or bundle branch block, supraventricular ectopy beat (S or SVEB), and ventricular ectopic beat (V or VEB) for each time-of-window.

After this analysis, a section that is not classified as one of the sections of the normal beat (N) or bundle branch block, the supraventricular ectopy beat (S or SVEB), and the ventricular ectopic beat (V or VEB) is classified as a noise section. When the noise section is classified through this process, it can take time first to classify the electrocardiogram signal as one of the normal beat (N) or bundle branch block, the supraventricular ectopy beat (S or SVEB), and the ventricular ectopic beat (V or VEB). That is, in order to detect a noise section, which does not require analysis, from an electrocardiogram signal, there is a problem that it takes time to analyze an electrocardiogram signal. In addition, when a preset time-of-window is determined to be a noise section, all signal segments included in the time-of-window may be determined to be noise and may be removed. Therefore, when a time interval of the time-of-window is increased to reduce the time to analyze an electrocardiogram signal, the amount of signal segments classified as a noise section increases, resulting in reduction in time to analyze the electrocardiogram signal excluding the noise section. However, when the time interval of the time-of-window is increased (lengthened), the time-of-window including the signal segments corresponding to the noise section may be classified as the noise section, and accordingly, all signal segments included in the time-of-window are classified as the noise section. That is, when a length of the time-of-window increases, the number of signal segments classified as a noise section increases, and there may be a problem that even signal segments, which do not correspond to the noise section, are removed. The time-of-window may be used in the same meaning as a window, and include may include one or more intervals and waveforms. The time-of-window may include one or more signal segments.

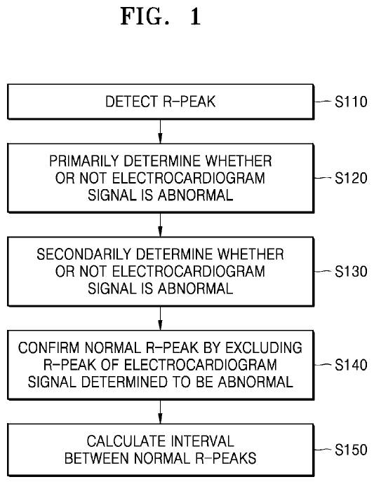

FIG. 1 is a flowchart illustrating a method of processing an electrocardiogram signal, according to an embodiment of the present disclosure.

As can be seen from FIG. 1, a method of processing an electrocardiogram signal, according to an embodiment of the present disclosure, includes an R-peak detection step S110 of detecting an R-peak of an electrocardiogram signal by using an electrocardiogram signal processing device; a first determination step S120 of primarily determining whether or not an electrocardiogram signal is abnormal by using the electrocardiogram signal processing device; a second determination step S130 of secondarily determining whether or not the electrocardiogram signal is abnormal based on a result of the first determination step S120 by using the electrocardiogram signal processing device; a confirmation step S140 of confirming a normal R-peak by excluding an R-peak of the electrocardiogram signal determined to be abnormal in the second determination step S130 by using the electrocardiogram signal processing device; and a step S150 of calculating an interval between the normal R-peaks confirmed in the confirmation step S140.

A method of processing an electrocardiogram signal may be performed by an electrocardiogram signal processing device. The electrocardiogram signal processing device may be implemented with hardware or software. The electrocardiogram signal processing device may be a computing device including one or more processors and may perform the method of processing an electrocardiogram signal by using the one or more processors. The electrocardiogram signal processing device may further include a memory. The memory may be electrically connected to the electrocardiogram signal processing device or may be connected to the electrocardiogram signal processing device through a network.

Here, the electrocardiogram signal may include one or more waveforms. For reference, the method of processing an electrocardiogram signal, according to an embodiment of the present disclosure, is performed on a preset detection region. In addition, the electrocardiogram signal used in the present disclosure is also referred to as a QRS signal.

Various known techniques of the related art including techniques of the related art described in the “Description of Related Art” may be used as the method of detecting an R-peak in the R-peak detection step S110, and thus, a separate description will not be made. The first determination step S120 may be implemented by two methods.

One is a method of using a time interval between R-peaks of an electrocardiogram signal, and the other is a method of using the complexity of the electrocardiogram signal.

The two methods may also be used in the first determination step S120 or may also be used in the first determination step S120 by being combined with each other. For reference, as an example of a combination of the two methods, when an electrocardiogram signal is determined to be an abnormal electrocardiogram signal by at least one of the two methods, it is determined that the electrocardiogram signal is abnormal or in an abnormal period in the first determination step S120.

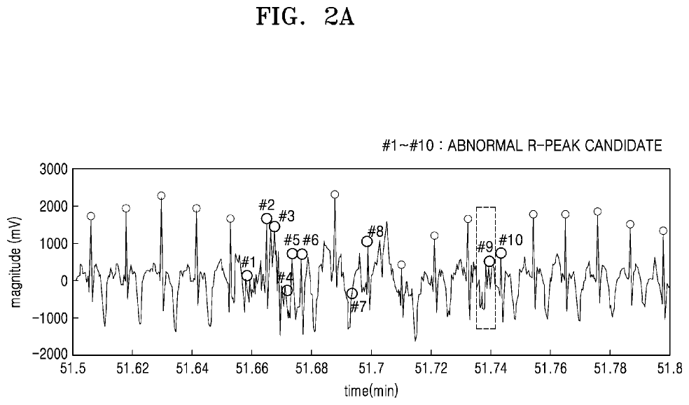

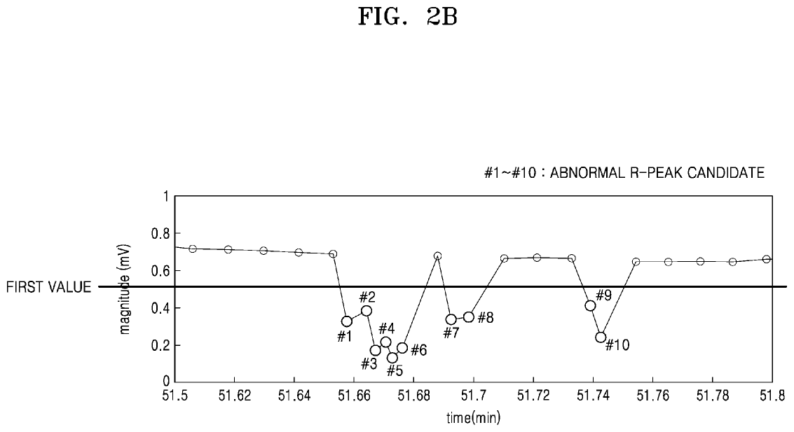

FIGS. 2A and 2B are explanatory diagrams of the first determination step S120 using a time interval between a first R-peak and a second R-peak of an electrocardiogram signal. The second R-peak may be referred to as a peak temporally adjacent to the first R-peak. FIG. 2A is a graph provided by extracting an R-peak from an electrocardiogram signal. FIG. 2B is an exemplary diagram of an abnormal R-peak in an electrocardiogram signal.

As illustrated in FIGS. 2A-2B, in a case in which an interval between the first R-peak and the second R-peak of the electrocardiogram signal is used, when the time interval (RR Interval) between the first R-peak and the second R-peak is less than a first value previously determined, the first R-peak may be primarily determined to be an abnormal R-peak in the first determination step S120. For reference, the R-peak determined as an abnormal R-peak becomes an R-peak that follows in time among the first R-peak and the second R-peak.

The first value may be set by using an average value of normal R-peaks of a normal electrocardiogram signal previously measured. For example, the first value may be set to 70% of the average value of the normal R-peaks.

The normal R-peak may be an R-peak, which is not an ideal R-peak.

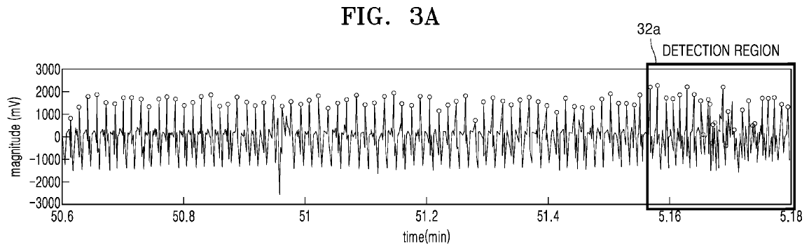

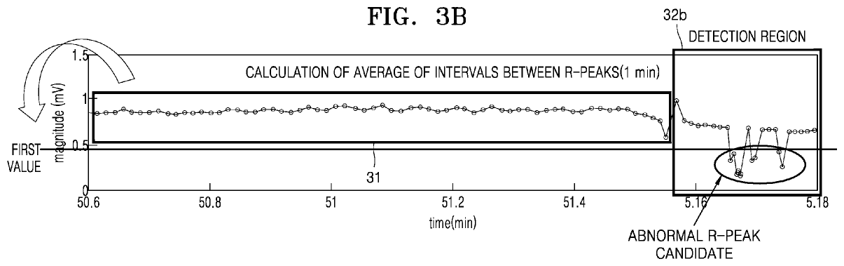

FIGS. 3A-3B are explanatory diagrams illustrating a method of setting a first value by using an average value of a normal R-peak value for a region of a normal electrocardiogram signal and a method of applying the first value to a detection region. FIG. 3A is an exemplary diagram of a detection region in an electrocardiogram signal. FIG. 3B is an exemplary diagram of an abnormal peak in a detection region in an electrocardiogram signal.

An electrocardiogram signal processing device may set the first value by using an average value of time intervals between normal R-peaks for a region 31 of the normal electrocardiogram signal. The electrocardiogram signal processing device may determine a candidate group of abnormal R-peaks by applying the first value to a detection region 32. Here, a normal electrocardiogram signal may be an electrocardiogram signal except for waveforms determined as an abnormal period (cycle or interval) in the electrocardiogram signal. Determination of an abnormal period may be performed by a disclosed method of the present disclosure but may be determined by various methods without being limited thereto. Here, the detection region may refer to all or part of the electrocardiogram signal and may refer to a region determined by a user. The first value refers to a value that is a criterion for determining an abnormal R-peak.

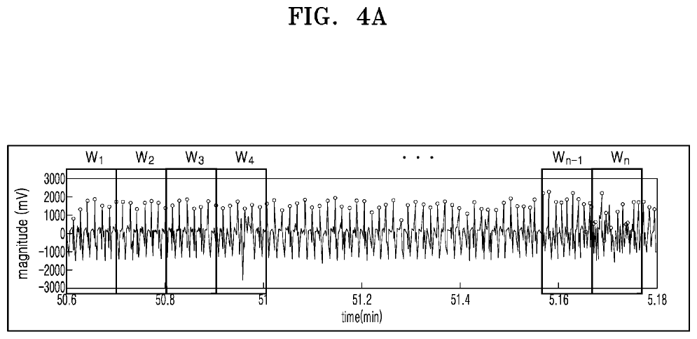

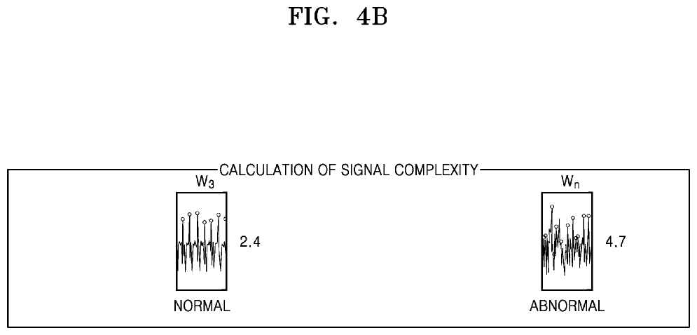

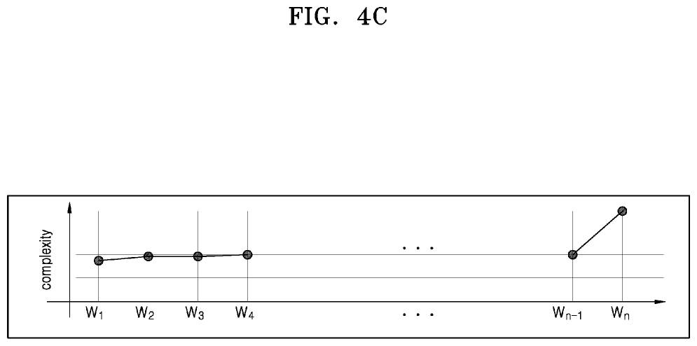

FIGS. 4A through 4C are explanatory diagrams illustrating a process of determining whether or not there is abnormality by using the complexity of an electrocardiogram signal. FIG. 4A is an exemplary diagram of windows of an electrocardiogram signal according to embodiments of the present disclosure. FIG. 4B is an exemplary diagram of a window including an abnormal peak extracted according to embodiments of the present disclosure. FIG. 4C is an exemplary diagram of complexity values calculated for windows of an electrocardiogram signal.

As illustrated in FIGS. 4A through 4C, in the first determination step S120, the complexity to which windows W1, W2, W3, W4, Wn−1, and Wn of a preset size (time interval) are applied is calculated for the electrocardiogram signal, and an electrocardiogram signal of a window out of a preset range is determined as an abnormal electrocardiogram signal. The abnormal electrocardiogram signal Wn may include one or more intervals and waveforms.

A method of calculating the complexity of a signal may include, for example, Shannon entropy, turning point ratio (TPR), and root mean square of the successive difference (RMSSD). Various known techniques may be used as the method of calculating the complexity of a signal, and thus, separate description will not be made.

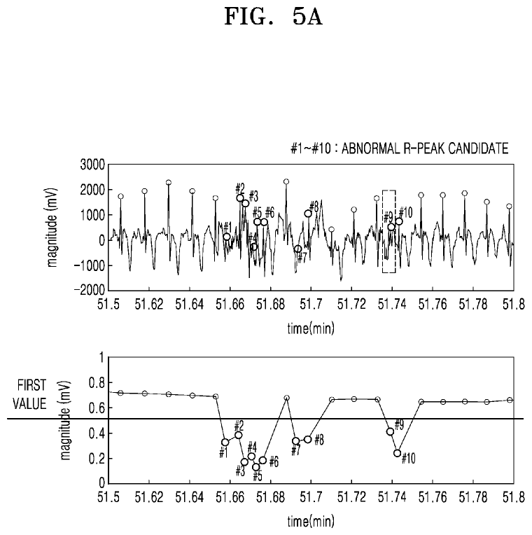

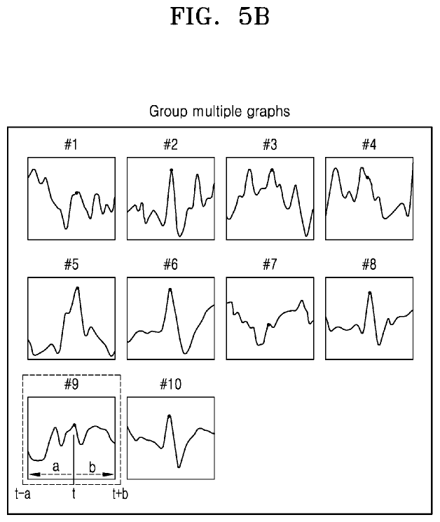

FIGS. 5A-5B are explanatory diagrams of waveforms of an abnormal electrocardiogram signal selected in the first determination step S120. FIG. 5A is an exemplary diagram of R-peaks of an electrocardiogram signal generated according to embodiments of the present disclosure. FIG. 5B is an exemplary diagram of signal waveforms of sections selected from an electrocardiogram signal.

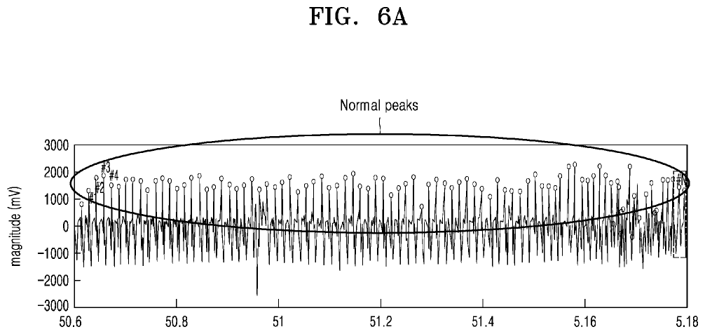

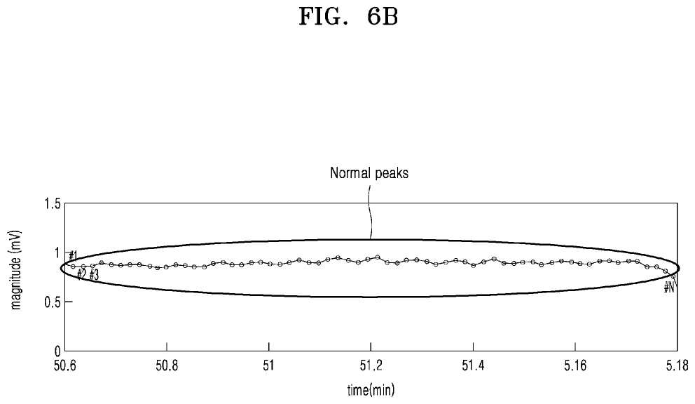

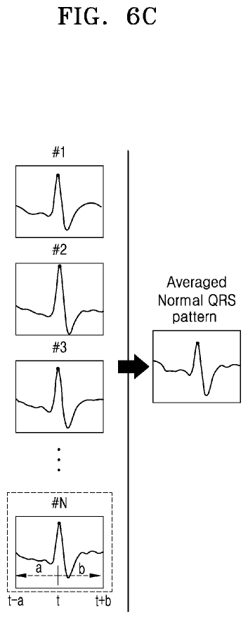

FIGS. 6A through 6C is an explanatory diagram illustrating a method of calculating an average value of waveforms of an electrocardiogram signal. FIG. 6A is an exemplary diagram of normal peaks extracted from an electrocardiogram signal. FIG. 6B is an exemplary diagram of peak interval values of normal peaks

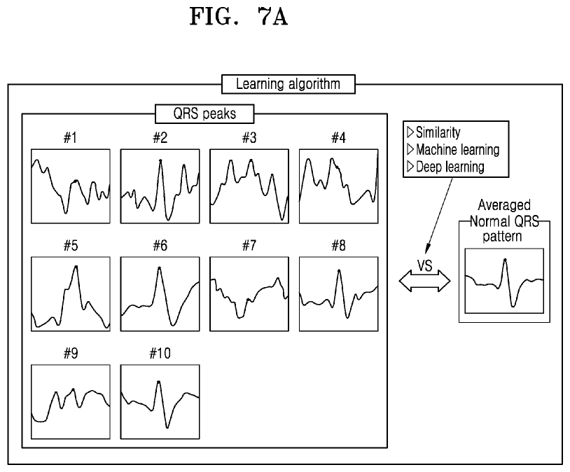

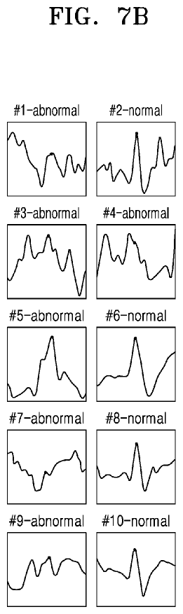

FIGS. 7A-7B is an explanatory diagram of a determination result of the second determination step S130. FIG. 7A depicts a group of QRS peaks and the averaged normal QRS pattern of FIG. 6C. FIG. 7B depicts abnormal and normal QRS peak patterns #1 through #10 of FIG. 7A.

The second determination step S130 will be described in detail with reference to FIGS. 5A through 7B.

In the second determination step S130, whether or not the electrocardiogram signal is abnormal is secondarily determined by comparing a waveform of the selected abnormal electrocardiogram signal with a waveform of a normal electrocardiogram signal by using an electrocardiogram signal processing device in the first determination step S120.

The waveform of the normal electrocardiogram signal is determined based on an average value of time intervals of waveforms of the electrocardiogram signal during a preset time period, in the second determination step S130. That is, a waveform of an electrocardiogram signal having N R-peaks may be included in the preset time period. As illustrated in FIGS. 6A-6B, the waveform of the electrocardiogram signal may include one pattern including a period from time t−a to time t+b, which is a preset region having a measurement time (or generation time) t of each R-peak as the center. The electrocardiogram signal processing device calculates an average value of time intervals between N waveforms of an electrocardiogram signal and desirably uses the average value of the time intervals of one or more waveforms of the electrocardiogram signal as a waveform of a normal electrocardiogram signal in the second determination step S130.

Specifically, in the second determination step S130, the electrocardiogram signal processing device compares each pattern with the waveform of the normal electrocardiogram signal by using the period from the time t-a to the time t+b, which is a preset region having a measurement time (or generation time) t of an R-peak of an abnormal electrocardiogram signal as the center in the first determination step S120.

The electrocardiogram signal processing device may also use artificial intelligence such as machine learning or deep learning or may also use a similarity determination method using feature points as a comparison method that may be used in the second determination step S130.

In the first determination step and the second determination step, whether or not an electrocardiogram signal is abnormal may be determined by using different criteria.

In the second determination step, the electrocardiogram signal processing device may determine whether or not the electrocardiogram signal is abnormal except for the abnormal period determined in the first determination step.

As can be seen from FIGS. 7A-7B, among ten abnormal electrocardiogram signals determined to be abnormal in the first determination step S110, the second electrocardiogram signal, the sixth electrocardiogram signal, the eighth electrocardiogram signal, and the tenth electrocardiogram signal are determined as normal electrocardiogram signals in the second determination step S130, and the first electrocardiogram signal, the third electrocardiogram signal, the fourth electrocardiogram signal, the fifth electrocardiogram signal, the seventh electrocardiogram signal, and the ninth electrocardiogram signal are finally determined as abnormal electrocardiogram signals in the second determination step S130.

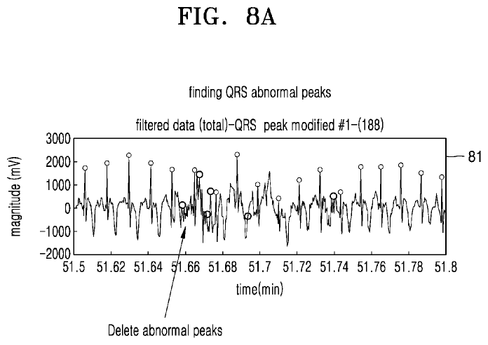

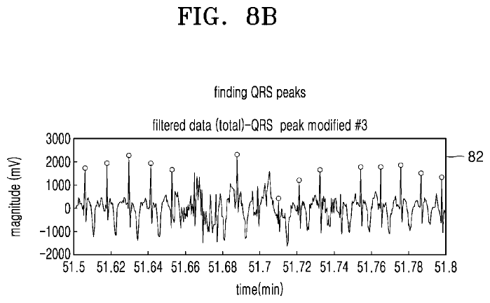

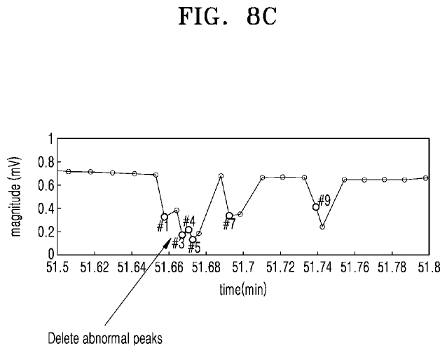

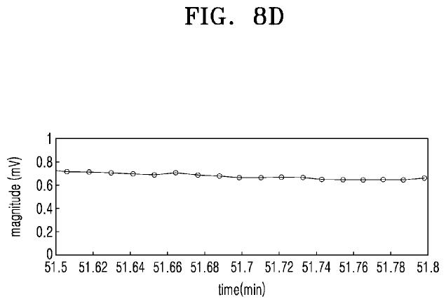

FIGS. 8A through 8D illustrates an explanatory diagram of the confirmation step S140. FIG. 8A is a diagram illustrating a process of deleting abnormal peaks from an electrocardiogram signal. FIG. 8B is a diagram illustrating a process of deleting abnormal peaks from an electrocardiogram signal. FIG. 8C is an exemplary diagram of peak values extracted from the electrocardiogram signal. FIG. 8D is an exemplary diagram of peak values excluding abnormal peaks from the ECG signal.

As can be seen from FIGS. 8A through 8D, in the confirmation step S140, the electrocardiogram signal processing device may determine normal R-peaks by excluding R-peaks of the electrocardiogram signal determined to be abnormal in the second determination step S130.

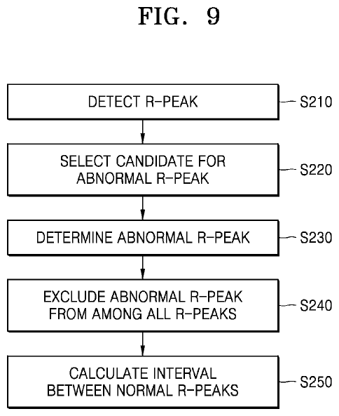

FIG. 9 is a flowchart when viewing, from another viewpoint, the flowchart of the method of processing an electrocardiogram signal according to the embodiment of the present disclosure illustrated in FIG. 1.

As can be seen from FIG. 9, the method of processing an electrocardiogram signal, according to the embodiment of the present disclosure, includes an R-peak detection step S210 of detecting an R-peak of an electrocardiogram signal by using an electrocardiogram signal processing device; a step S220 of selecting candidates for an abnormal R-peak from the electrocardiogram signal by using the electrocardiogram signal processing device; a step S230 of determining an abnormal R-peak among the candidates selected by the electrocardiogram signal processing device in step S220; a step S240 of excluding the abnormal R-peak determined in step S230 among all of the R-peaks from the electrocardiogram signal; and a step of calculating an interval between normal R-peaks confirmed by the electrocardiogram signal processing device in step S240.

In step S220, at least one time interval between R-peaks of an electrocardiogram signal, and complexity of the electrocardiogram signal may be used.

Specifically, when using the time interval between the R-peaks of the electrocardiogram signal, step S220 includes step S221a of calculating an interval between the R-peaks of the electrocardiogram signal; and step S333a of selecting the corresponding R-peak as a candidate for an abnormal R-peak when the interval between the R-peaks calculated in step S221a is less than a preset value.

In addition, when using the complexity of the electrocardiogram signal, step S220 desirably includes step S221b of calculating the complexity of a signal to a preset window size; and step S222b of selecting an R-peak included in a window in which the complexity of the signal calculated in step S221b is out of a preset range, as a candidate for an abnormal R-peak.

In addition, in step S230, an abnormal R-peak is determined from among the candidates selected in step S220 by comparing a waveform of an electrocardiogram signal during a time period previously set based on the time representing the R-peak selected in step S220 with a waveform of an electrocardiogram signal representing a normal R-peak. The waveform of the electrocardiogram signal representing the normal R-peak may be determined by machine learning learned from waveforms of electrocardiogram signals or a waveform of an electrocardiogram signal in the past record of the patient. The waveform of the electrocardiogram signal measured by the patch-type measuring device may have a different shape depending on the attachment position. The waveform of the electrocardiogram signal representing the normal R-peak can be different for each measurement.

In addition, the waveform of the electrocardiogram signal representing the normal R-peak may be calculated by an average value of time intervals of waveforms of the electrocardiogram signal for multiple R-peaks during a preset time period.

As described above, according to the method of processing an electrocardiogram signal, according to the present disclosure, an electrocardiogram signal processing device detects a candidate for an abnormal electrocardiogram signal in S220, and the abnormal electrocardiogram signal may be finally and accurately determined in S230. In addition, when an abnormal electrocardiogram signal is determined in S230 without being processed as in step S220, the amount of calculations is increased, and determination of the abnormal electrocardiogram signal due to a small amount of calculations may be made by adding step S220.

That is, according to the method of processing an electrocardiogram signal, according to the present disclosure, it can be seen that an abnormal electrocardiogram signal may be accurately determined by determining whether or not an electrocardiogram signal is abnormal through the S220 and S230.

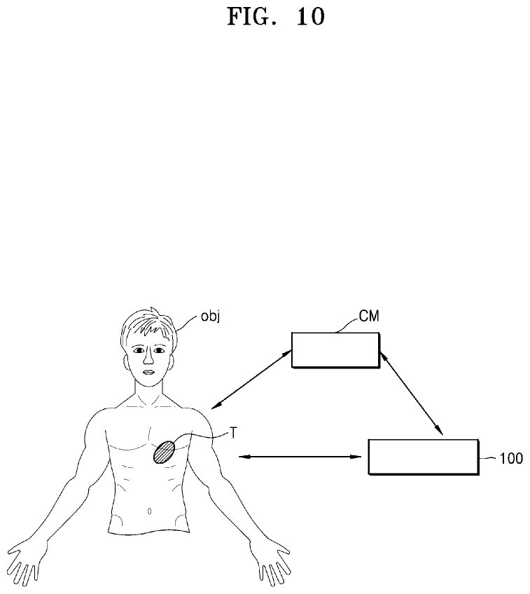

FIG. 10 is a view illustrating a network environment of an electrocardiogram measurement device T according to embodiments.

As illustrated in FIG. 10, the electrocardiogram measurement device T may be non-invasively or invasively mounted on an object obj to detect an electrocardiogram according to a heartbeat of the object obj. Here, the object obj may be a human body or an animal body, such as a human chest or an animal chest but is not limited thereto, and any object may be the object as long as an electrocardiogram may be detected or measured. The electrocardiogram measurement device T may receive and process a measured electrocardiogram signal by using a preset method. A program related to signal processing may be stored in the electrocardiogram measurement device T to be executed.

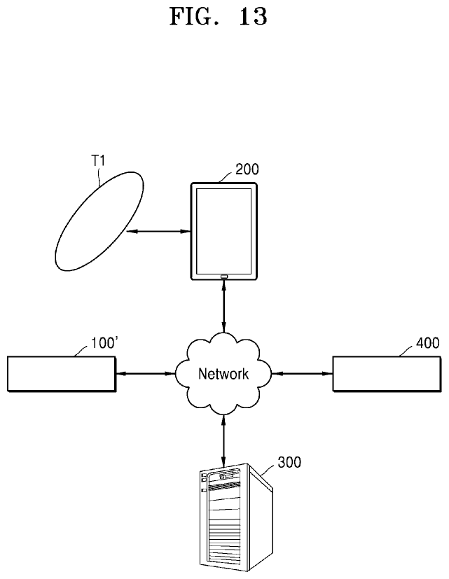

The electrocardiogram measurement device T may transmit and receive data to and from a user terminal 200 (see FIG. 13) by using a communication unit. The communication unit may include various communication modules, such as a wireless Internet module, a near field communication module, and a mobile communication module.

The wireless Internet module may be connected to an external network to communicate therewith according to communication protocols, such as wireless local area network (WLAN), Wi-Fi, wireless broadband (Wibro), world interoperability for microwave access (Wimax), and high speed downlink packet access (HSDPA).

The near field communication module communicates with external devices located in a short distance according to a near field communication method, such as Bluetooth, radio frequency identification (RFID), infrared data association (IrDA), ultrawideband (UWB), and ZigBee.

The mobile communication module performs communication by accessing a mobile communication network according to various mobile communication standards, such as third generation (3G), third generation partnership project (3GPP), and long term evolution (LTE).

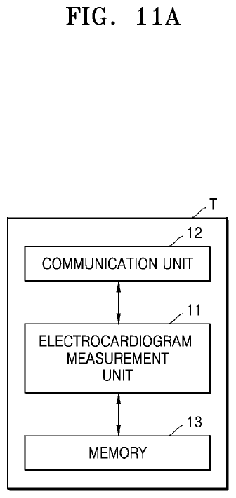

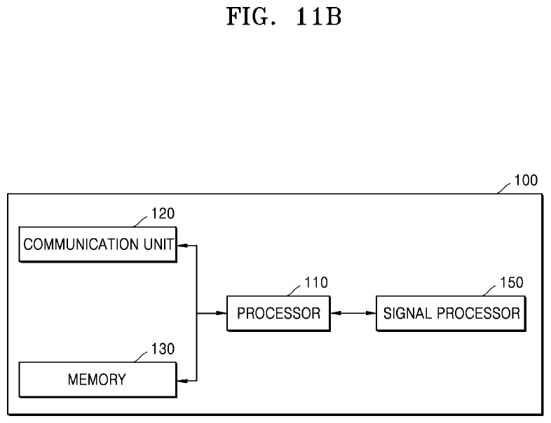

However, the present disclosure is not limited thereto, and as long as a communication unit 120 (see FIG. 11B) may communicate with the electrocardiogram measurement device T and transmit and receive various signals and data, the communication unit 120 may include communication modules that are different from the components described above.

The electrocardiogram measurement device T may further include a mounting portion. The mounting portion may include a flexible material that may be deformed according to a curved body surface, for example, elastic cloth, that is, stretchable cloth. The mounting portion may be provided in a patch type or a wearable type. Due to being worn through the mounting portion, the electrocardiogram measurement device T may come into contact with a body surface of an object to detect an electric potential generated on the body surface.

The electrocardiogram measurement device T may include one or more measurement electrodes for measuring an electrocardiogram signal. The electrocardiogram measurement device T may further include a heart rate sensor, a respiration sensor, a temperature sensor, a heart sound sensor, and so on that measure other detection signals as needed. The electrocardiogram measurement device T may store the measured electrocardiogram signal in an internal memory thereof. The electrocardiogram measurement device T may transmit an electrocardiogram signal to an external device, for example, an electrocardiogram signal processing device 100 through a communication unit (see 12 in FIG. 11A) of the electrocardiogram measurement device T directly or via a communication means CM. The electrocardiogram measurement device T may transmit the measured electrocardiogram signal to the electrocardiogram signal processing device 100 in real time or transmit the stored electrocardiogram signal to the electrocardiogram signal processing device 100.

The electrocardiogram signal processing device 100 may divide the electrocardiogram signal obtained by measuring the object obj into preset signal segments and group or cluster the signal segments. The electrocardiogram signal processing apparatus 100 may divide the electrocardiogram signal into signal segments by dividing the electrocardiogram signal according to a division rule determined by R interval or R peak. The electrocardiogram signal processing device 100 may group or cluster signal segments having a similar pattern to each reference signal segment based on one or more reference signal segments. The electrocardiogram signal processing device 100 may group or cluster signal segments using an electrocardiogram classification model. The electrocardiogram classification model may be learned by deep learning or machine learning. The electrocardiogram classification model may be learned by using measured electrocardiogram signals as input. The electrocardiogram signal processing device 100 may group or cluster signal segments corresponding to each reference value based on one or more reference values.

FIG. 11A is a block diagram of the electrocardiogram measurement device T according to embodiments, and FIG. 11B is a block diagram of the electrocardiogram signal processing device 100 according to embodiments.

As illustrated in FIG. 11A, the electrocardiogram measurement device T may include an electrocardiogram measurement unit 11, a communication unit 12, and/or a memory 13. The electrocardiogram measurement device T may further include other components, such as a respiratory rate sensor and a motion sensor. The electrocardiogram measurement device T may be attached to the object obj to measure an electrocardiogram signal and store the measured electrocardiogram signal. The electrocardiogram measurement device T may transmit an electrocardiogram signal to an external device. The electrocardiogram measurement device T may transmit analysis data acquired by processing the electrocardiogram signal to the external device.

The communication unit 12 may communicate with various types of external devices or servers according to various types of communication methods. The communication unit 12 may be connected to a communication unit of another device through a network to exchange data therewith. A program of the electrocardiogram measurement unit 11 or a program stored in the memory 13 may be received through the communication unit 12 and installed therein. The electrocardiogram measurement device T may transmit an electrocardiogram signal measured by the communication unit 12 to an external device. The communication unit 12 may include a device for wireless communication, such as a Wi-Fi chip, a Bluetooth chip, a wireless communication chip, or a near field communication (NFC) chip. The wireless communication chip refers to a chip that performs communication according to various communication standards, such as institute of electrical and electronics engineers (IEEE), Zigbee, 3G, 3GPP, and LTE. The NFC chip refers to a chip that operates in an NFC method using a band of 13.56 MHz among various radio frequency identification (RFID) frequency bands, such as 135 kHz, 13.56 MHz, 433 MHz, 860 to 960 MHz, and 2.45 GHz. In another embodiment, the communication unit 12 may include a device for wired communication using a communication line to exchange data with an external device. The device for wired communication may include a network interface controller and so on.

The memory 13 may store data related to electrocardiogram measurement. The memory 13 may store various data processed by the electrocardiogram measurement device T and store data processed by the processor 11 and data to be processed. The memory 13 may be implemented by random access memory (RAM), such as dynamic random-access memory (DRAM) or static random access memory (SRAM), read-only memory (ROM), electrically erasable programmable read-only memory (EEPROM), and flash memory.

The electrocardiogram measurement unit 11 may output an electrocardiogram signal by detecting an electrical signal output from the heart of an object. The electrocardiogram measurement unit 11 may include electrodes for measuring potentials that appear on a body surface in relation to heartbeat or for applying a current to inject electrolyte drugs into the body through a user's skin or mucous membranes The electrodes may include two or more electrodes. The electrocardiogram measurement unit 11 may output an electrocardiogram signal by measuring impedance between electrodes. The electrocardiogram measurement unit 11 may further include a shielding layer that comes into contact with a user's skin to cause external static electricity to flow to the user's skin. The electrocardiogram measurement unit 11 may further include a ground electrode. The electrocardiogram measurement unit 11 may be implemented in the form of an electrocardiogram patch. The electrocardiogram measurement unit 11 may include electrodes for measurement and a chip in which firmware software for processing signals from the electrodes is stored.

An electrocardiogram signal measured by the electrocardiogram measurement device T of a patch type may have a different signal pattern for each object. Also, the electrocardiogram signal may have a different signal pattern for each position (e.g. location) where the electrocardiogram measurement device T is attached.

The electrocardiogram measurement unit 11 may segment the measured electrocardiogram signal. The electrocardiogram measurement unit 11 may normalize an electrocardiogram signal.

FIG. 11B is a block diagram of the electrocardiogram signal processing device 100 according to embodiments.

As illustrated in FIG. 11B, the electrocardiogram signal processing device 100 may include a processor 110, a communication unit 120, a memory 130, and/or a signal processor 150.

The processor 110 may perform all operations for controlling the electrocardiogram signal processing device 100 by using various programs stored in the memory 130. The processor 110 may perform all operations for generally controlling the electrocardiogram signal processing device 100 by using a program stored in the signal processor 150. The processor 110 may include a microprocessor, a central processing unit (CPU), a processor core, a multiprocessor, an application-specific integrated circuit (ASIC), and a field programmable gate array (FPGA) but is not limited thereto.

The communication unit 120 may communicate with various types of external devices or servers according to various types of communication methods. The communication unit 120 may be connected to a communication unit of another device through a network to exchange data therewith. A program of the signal processor 150 and/or a program stored in the memory 130 may be received through the communication unit 120 and installed therein. The electrocardiogram signal processing device 100 may transmit an electrocardiogram signal measured through the communication unit 120 and a signal of an analysis target section obtained by removing noise from the electrocardiogram signal to an external device. The communication unit 120 may include a device for wireless communication, such as a Wi-Fi chip, a Bluetooth chip, a wireless communication chip, or an NFC chip. The wireless communication chip performs communication according to various communication standards, such as IEEE, Zigbee, 3G, 3GPP, and LTE. The NFC chip refers to a chip that operates in an NFC method using a band of 13.56 MHz among various radio frequency identification (RFID) frequency bands, such as 135 kHz, 13.56 MHz, 433 MHz, 860 to 960 MHz, and 2.45 GHz. In another embodiment, the communication unit 130 may include a device for wired communication using a communication line to exchange data with an external device. The device for wired communication may include a network interface controller and so on.

The memory 130 may store data related to electrocardiogram measurement. The memory 130 may store various types of data processed by the electrocardiogram signal processing device 100 and store data processed by the processor 110. The memory 130 may be implemented by RAM, such as DRAM or SRAM, ROM, EEPROM, and flash memory.

The measured electrocardiogram signal may have a different signal pattern for each object. Also, the electrocardiogram signal may have a different signal pattern for each position where the electrocardiogram signal processing device 100 is attached.

The electrocardiogram signal processing device 100 may receive an electrocardiogram signal from at least one of the electrocardiogram measurement device T, a data storage device, and a cloud computing device.

The signal processor 150 may process an electrocardiogram signal and classify a noise section and an analysis target section of the electrocardiogram signal. The signal processor 150 may be implemented as firmware and may be a storage medium and/or memory in which a program for processing an electrocardiogram signal is recorded.

The signal processor 150 may group or cluster electrocardiogram signals to classify signal segments of the electrocardiogram signals into one or more groups and set signal segments not classified into an abnormal groups. The signal processor 150 may group or cluster electrocardiogram signals using deep learning or machine learning. Among the signal segments of the abnormal group, a signal segment satisfying a preset criterion may be set as a noise section. The signal processor 150 may determine the noise section among the signal segments of the abnormal group using a noise decision model. A noise decision model may be a model for determining the noise section using the preset criterion. Signal segments that do not belong to the noise section may be classified as an analysis target section. The signal processor 150 may classify signal segments belonging to the one or more groups and signal segment belonging to the abnormal group but not belonging to the noise section as an analysis target period.

Grouping and/or clustering the electrocardiogram signals may be performed based on signal patterns of the electrocardiogram signals. An electrocardiogram signal may include repetition of similar patterns of signal segments. Signal segments deviating from similar patterns may correspond to the one or more groups or the abnormal group.

The signal processor 150 may analyze a pattern of reference signal segments of an electrocardiogram signal using deep learning or machine learning and classify signal segments deviating from a similar pattern. According to embodiments, The signal processor 150 may determine each group of each signal segments based on comparison with one or more reference signal segments or comparison with one or more reference values. The one or more reference values may be one or more similarity values or one or more complexity values.

The signal processor 150 may perform segmentation before classifying an electrocardiogram signal. The signal processor 150 may normalize an electrocardiogram signal. Normalizing a signal may be performed by changing peak values of an electrocardiogram signal and amplitudes of the peak values to relative values. Changing to the relative value may mean changing to a relative size within a period of a signal.

In another embodiment, the signal processor 150 may normalize signal segments of an electrocardiogram signal. Accordingly, classification may be performed according to relative values rather than absolute values. The signal processor 150 may divide an electrocardiogram signal into signal segments by segment the electrocardiogram signal. For example, the signal segment may include one PQRST or a preset number of PQRSTs.

More specifically, the signal processor 150 may segment the electrocardiogram signal based on a pattern. Here, the pattern of the electrocardiogram signal may be determined using at least one of peaks, P peaks, R peaks, and QRS time intervals of the electrocardiogram signal. The signal processor 150 may segment an electrocardiogram signal based on intervals of peaks, or magnitudes of peaks. The signal processor 150 may segment an electrocardiogram signal based on the QRS time interval. Accordingly, an electrocardiogram signal may be divided into signal segments after segmenting.

The signal processor 150 may set signal segments of an electrocardiogram measured as a total set. The signal processor 150 may classify the signal segments into one or more groups. The signal processor 150 may classify signal segments not belonging to the one or more group into an abnormal group.

The signal processor 150 may determine signal segments as a noise section using a noise decision model among signal segments that belong to the abnormal group. For example, the signal processor 150 may determine, as a noise section, two or more signal segments measured consecutively among signal segments belonging to an abnormal group. The signal processing unit 150 may set signal segments that are not noise sections as the analysis target section. The noise decision model may be learned by taking electrocardiogram signals in a noise section and electrocardiogram signals not in a noise section as inputs. The noise decision model may be learned using deep learning or machine learning.

The signal processor 150 may classify signal segments into one or more groups using any one of signal segments. The signal processor 150 may select a preset number of reference signal segments from among signal segments and may group or cluster the signal segments by using similarity values with the reference signal segments. For example, the signal processor 150 may select a first reference signal segment, a second reference signal segment, and a third reference signal segment from among signal segments. The signal processor 150 may classify, as a first group, signal segments having a correlation coefficient that is greater than or equal to a reference similarity value compared to the first reference signal segment, classify, as a second group, signal segments having a correlation coefficient that is greater than or equal to the reference similarity value compared to the second reference signal segment, and classify, as a third group, signal segments having a correlation coefficient that is greater than or equal to the reference similarity value compared to the third reference signal segment. The signal processor 150 may classify signal segments that do not belong to the first group, the second group, and the third group as the abnormal group. The signal processor 150 may determine temporally adjacent signal segments among signal segments included in the abnormal group as a noise section. In the above description, three reference signal segments are selected, but this is only an example, and the number of selected reference signal segments may be changed to two, five, or more. The number of selected reference signal segments may be changed by considering a pattern of signal segments included in an electrocardiogram signal.

Here, the first reference signal segment, the second reference signal segment, and/or the third reference signal segment may be selected based on the frequency of occurrence. Or, the first reference signal segment, the second reference signal segment, and/or the third reference signal segment may be determined using deep learning or machine learning.

The signal segments classified as the abnormal group may be signal segments that are not classified into the one or more groups as a result of classification by the electrocardiogram classification model.

In another embodiment, the signal processor 150 may classify signal segments into the one or more groups and the abnormal group based on a complexity value, a time interval value, a peak value and/or so on. The complexity value, a time interval value, a peak value and/or so on is determined using the electrocardiogram classification model. More specifically, the signal processor 150 may determine complexity values as a first reference value, a second reference value, and/or a third reference value in descending order of the frequency of occurrence, classify signal segments having a complexity value of the first reference value as a first group, classify signal segments having a complexity value of the second reference value as a second group, classify signal segments having a complexity value of the third reference value as a third group, and classify signal segments that do not belong to the first group, the second group, and the third group as an abnormal group.

Here, the signal segment having a complexity value of the first reference value may refer to a signal segment having a complexity value in a range related to the first reference value. The range related to the first reference value may be set to a value that is less than or equal to the first reference value, a value that is greater than or equal to the first reference value, a value of which difference from the first reference value is less than or equal to a preset difference value, or so on, but is not limited thereto and may be set in various ranges.

In another embodiment, the first complexity value to the third complexity value may be a complexity value of a first signal segment, a complexity value of a second signal segment, and/or a complexity value of a third signal segment of a pattern with a high frequency of occurrence.

The signal processor 150 may classify temporally adjacent signal segments among signal segments included in an abnormal group as a noise section. In the above description, the first reference value, the second reference value, and/or the third reference value may be determined as time interval values or peak values, and signal segments may be classified by using reference values that are the time interval values or the peak values.

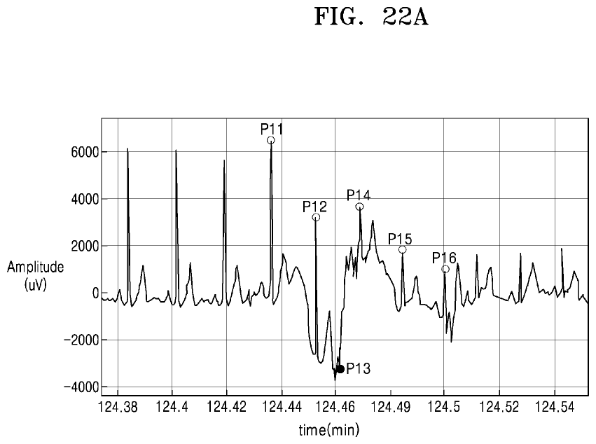

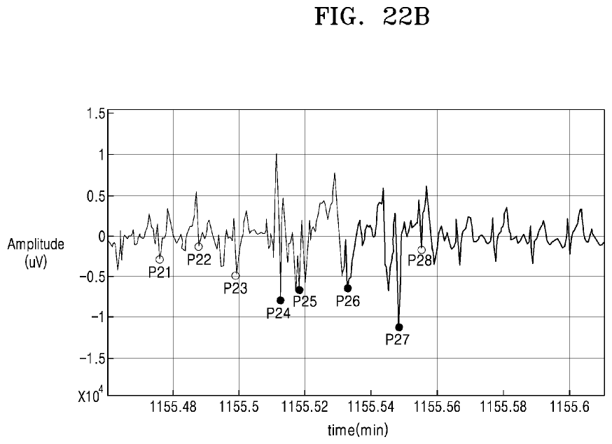

The signal processor 150 may determine whether or not signal segments classified as the abnormal group correspond to a noise section using the noise decision model. The signal processor 150 may determine whether signal segments classified as the abnormal group are temporally adjacent signal segments. As a result of the clustering, the signal processor 150 may insert group information of each of the signal segments into an electrocardiogram signal. Data in which group information is inserted into an electrocardiogram signal may be as illustrated in FIGS. 22A and 22B.

The signal processor 150 may generate data by listing group information of signal segments of an electrocardiogram signal in a time dimension. The signal processor 150 may scan group information of signal segments to detect the first signal segment classified as the abnormal group and may determine whether the second signal segment subsequent to the first signal segment corresponds to the abnormal group. When both the first signal segment and the second signal segment correspond to the abnormal group, the first signal segment and the second signal segment may be classified as a noise section. When the second signal segment corresponds to the one or more group, the first signal segment may not be classified as an abnormal section.

The electrocardiogram signal processing device 100 may generate output data for displaying the electrocardiogram signal excluding the noise section. The electrocardiogram signal processing apparatus 100 may generate data for signal segments belonging to the one or more groups. The electrocardiogram signal processing apparatus 100 may generate data for signal segments belonging to the abnormal group. The electrocardiogram signal processing apparatus 100 may input signal segments to a machine learning model and/or a deep learning model related to electrocardiogram signal processing in order to train models related to electrocardiogram signal processing. The electrocardiogram signal processing device 100 may analyze each signal segment of the electrocardiogram signal from which a noise section is removed. The electrocardiogram signal processing device 100 may analyze each signal segment as one of a normal beat (N) or bundle branch block), a supraventricular ectopy beat (S, SVEB) and a ventricular ectopic beat (V, VEB). The electrocardiogram signal processing device 100 may generate data related to the analysis target section for each signal segment. The electrocardiogram signal processing device 100 may analyze the electrocardiogram signal measured by the electrocardiogram measurement device T and classify signal segments corresponding to a noise section. The electrocardiogram signal processing device 100 may classify the electrocardiogram signal excluding the noise section into an analysis target section. By preferentially classifying the electrocardiogram signal in the noise section and analyzing only the electrocardiogram signal in the analysis target section, time and/or resource for analyzing the electrocardiogram signal in the noise section can be saved.

In the above description, the electrocardiogram signal processing device 100 is described as a separate device, but it is natural that the electrocardiogram signal processing device 100 may be implemented in the user terminal 200, an electrocardiogram management server 300, a computing device 400, and so on.

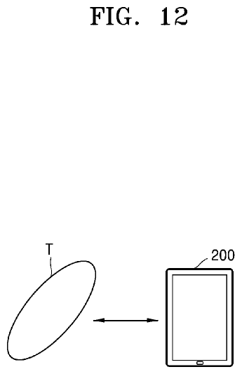

FIG. 12 illustrates a network environment of the electrocardiogram measurement device T according to embodiments.

As illustrated in FIG. 12, the electrocardiogram measurement device T may exchange data with the user terminal 200. Here, the user terminal 200 may be a mobile phone, a smartphone, a laptop computer, a digital broadcast terminal, a personal digital assistant (PDA), or a portable multimedia player (PMP) but is not limited thereto, and any mobile device may be used, and the terms terminal and device may be used interchangeably.

The electrocardiogram measurement device T may transmit a measured electrocardiogram signal to the user terminal 200. The electrocardiogram measurement device T may transmit an electrocardiogram signal and information on the measurement time of the electrocardiogram signal to the user terminal 200. The information on the measurement time may include at least one of a measurement start time and a measurement end time. The electrocardiogram measurement device T may transmit information on an object to the user terminal 200. The information on the object may include at least one of a name, an age, and a gender of the object.

The electrocardiogram measurement device T may start or end measurement of an electrocardiogram signal in response to a measurement start signal or a measurement end signal from the user terminal 200. The electrocardiogram measurement device T may remove data recorded in a memory in response to a data removal signal from the user terminal 200.

The user terminal 200 communicating with the electrocardiogram measurement device T may be registered through a preset registration process.

The user terminal 200 may receive an electrocardiogram signal measured by the electrocardiogram measurement device T. The user terminal 200 may be carried by an object that measures an electrocardiogram signal. When an object feels pain for the heart, a preset input may be input to the user terminal 200. In this case, the user terminal 200 may receive an electrocardiogram signal of a time section including an input time of pain from the electrocardiogram measurement device T. The user terminal 200 may display the electrocardiogram signal received in this way. The user terminal 200 may separately store the electrocardiogram signal at the moment of pain in response to one or more inputs of pain.

The user terminal 200 may receive an electrocardiogram signal measured in real time when the electrocardiogram measurement device T starts measurement. The user terminal 200 may display an electrocardiogram signal. The displayed electrocardiogram signal may be used to determine whether an attachment position of the electrocardiogram measurement device T is suitable. When the electrocardiogram signal displayed on the user terminal 200 does not have a normal pattern, it can be determined that the attachment state of the electrocardiogram measurement device T is not suitable. The normal pattern may be determined according to pre-stored pattern information. When the electrocardiogram signal has a certain regularity, the electrocardiogram signal may be determined to have a normal pattern. When the signal interval between signal values of an electrocardiogram signal is constant or when magnitudes of signal values of the electrocardiogram signal are periodically repeated, the electrocardiogram signal may be determined to have regularity. The user terminal 200 may display a message on an electrocardiogram signal that does not have a normal pattern.

A target subject or a medical staff of the object may check suitability of an attachment position of the electrocardiogram measurement device T while viewing an electrocardiogram signal displayed on the user terminal 200. When an electrocardiogram signal that does not have a normal pattern is displayed, the attachment position of the electrocardiogram measurement device T may be changed.

In another embodiment, the user terminal 200 may be an electronic device or a wired/wireless hub carried by a medical staff associated with an object.

FIG. 13 illustrates an another embodiment of FIG. 10. Here, an electrocardiogram signal processing device 100′ may be a device including the same components as the electrocardiogram signal processing device 100 (see FIG. 11B). The electrocardiogram signal processing device 100′ may further include additional components. FIG. 10 illustrates a case of a small hospital where ECG analysis and ECG diagnosis can be performed in one entity, while FIG. 13 illustrates a cooperative working case of separated experts of ECG analysis, diagnosis including ECG, management of electronic medical record, etc. AI and/or machine learning may be applied to the electrocardiogram signal processing device 100′ connected to cloud networking to diagnose and predict ECG-related diseases.

As illustrated in FIG. 13, an electrocardiogram signal measured by the electrocardiogram measurement device T1 may be transmitted to the electrocardiogram signal processing device 100′ through the user terminal 200. Electrocardiogram signals measured by one or more electrocardiogram measurement devices T1 may be collected in the electrocardiogram management server 300 through one or more user terminals 200. The electrocardiogram signal collected in this way may be classified into a noise section and an analysis target section in the electrocardiogram signal processing apparatus 100′. A diagnostician, an analyst, etc. may receive the data processed by the electrocardiogram signal processing device 100′ and determine an ECG-related disease of the subject. Data generated by the ECG signal processing device 100′ may be transmitted to and managed by the ECG management server 300. A diagnostician, an analyst, etc. may access the electrocardiogram signal processing device 100′ or the electrocardiogram management server 300 through a separate computing device 400 to check electrocardiogram-related data and input ECG-related disease information.

The electrocardiogram measurement device T1 may be a portable electrocardiogram recording device, such as an adhesive holder patch, an electrocardiogram sensor, an event recorder, or a mobile telemetry. The user terminal 200 may communicate with the electrocardiogram measurement device T1 through a wireless or wired communication network, such as Bluetooth or Bluetooth low energy (BLE).

The user terminal 200 may check an emergency situation of an object and perform analysis on an electrocardiogram signal received to directly send an alarm to a clinical staff when the emergency situation is detected. In the emergency situation, a patient's attending physician may tell a general or predefined arrhythmia event type and duration to a certain patient. The user terminal 200 may compare the measured electrocardiogram signal with a pre-registered event type and determine whether an emergency situation of an object occurs. When it is determined that the emergency situation occurs, the user terminal 200 may transmit a message to a terminal of a clinical staff or so on. The user terminal 200 may transmit an instruction signal for additional analysis by transmitting an electrocardiogram signal of an emergency situation to a terminal of a clinical staff or so on.

The user terminal 200 may analyze the electrocardiogram signal to determine whether a pre-stored event type or trigger occurs from the electrocardiogram signal.

The user terminal 200 may determine whether an emergency situation occurs and whether a trigger occurs by comparing a signal pattern of an emergency situation and a signal pattern corresponding to the trigger with an electrocardiogram signal.

The user terminal 200 may transmit data for an electrocardiogram signal determined as occurrence of an emergency to the electrocardiogram signal processing device 100 or the electrocardiogram management server 400. The user terminal 200 may transmit data for an electrocardiogram signal corresponding to a pre-stored event type or trigger to the electrocardiogram signal processing device 100′ or the electrocardiogram management server 400.

The user terminal 200 may transmit data of a predetermined period, and data, such as a patient's trigger or an algorithm trigger to either the electrocardiogram signal processing device 100′ or the electrocardiogram management server 400. Also, the electrocardiogram data of an electrocardiogram measurement device T1 may be real-time data or data stored in the memory 13.

The electrocardiogram signal processing device 100′ may receive an electrocardiogram signal, information on measurement time, and/or information on an object from the user terminal 200 through a network, such as Wi-Fi, the Internet, a mobile data network, a cellular network, or a telephone line connection. The electrocardiogram signal processing device 100′ may receive an electrocardiogram signal, data related to the analysis target section of the electrocardiogram signal, object information, measurement time point information, measurement device information, and so on from at least one of the user terminal 200, the electrocardiogram management server 300, and the computing device 400.

The electrocardiogram management server 300 may communicate with at least one of the electrocardiogram signal processing device 100′, the user terminal 200, and the computing device 400 through a network, such as the Internet, a mobile data network, a cellular network, or a telephone line connection. The electrocardiogram management server 300 may be implemented in a distributed manner. The electrocardiogram management server 300 may be operated by a virtual storage solution installed therein.

The electrocardiogram management server 300 may receive an electrocardiogram signal and/or data for the electrocardiogram signal from the user terminal 200. The electrocardiogram management server 300 may receive an electrocardiogram signal and/or data for the electrocardiogram signal from a plurality of user terminals 200. The electrocardiogram management server 300 may receive, from the electrocardiogram signal processing device 100′, data obtained by removing a noise section from an electrocardiogram signal and data for a result of classifying the electrocardiogram signal. The electrocardiogram management server 300 may store and manage the received data. The electrocardiogram management server 300 may upload and manage data, such as a measured electrocardiogram signal, measurement time information, and object information. In an optional embodiment, the electrocardiogram management server 300 may differentially manage a received electrocardiogram signal and/or data for the electrocardiogram signal according to an object, a measurement time point, and/or a measurement device.

The electrocardiogram management server 300 may download data stored according to a request of the computing device 400 into the computing device 400. The electrocardiogram management server 300 may determine a price for each transmitted data and request payment of a preset cost to the computing device 400 or an account of the computing device 400. A price for each data may be set in proportion to the amount of resources required for processing. The electrocardiogram management server 300 may transmit an electrocardiogram signal and/or an electrocardiogram signal from which a noise section is removed, in response to a request signal. In addition, data processed in various ways may be transmitted.

The electrocardiogram management server 300 may manage data received from the electrocardiogram signal processing device 100′ in association with an object and/or a measurement time point. The electrocardiogram management server 300 may store an electrocardiogram signal measured for a first object and data for the electrocardiogram signal in relation to the first object and/or the first measurement time point.

In transmitting data, the electrocardiogram management server 300 may be designed to provide data of the first object to a user having authority for the first object. A user having authority for the first object may be at least one of a medical worker, a legal person in charge, and a person employed for management. The electrocardiogram management server 300 may transmit electrocardiogram data of a target object requested by a medical worker.

In an optional embodiment, the electrocardiogram management server 300 may store and manage a signal processing solution of the electrocardiogram signal processing device 100. The signal processing solution may determine a reference signal segment and/or a reference value for grouping electrocardiogram signals. The electrocardiogram management server 300 may transmit and update the signal processing solution to the electrocardiogram signal processing device 100′. The electrocardiogram management server 300 may search for stored data.

The electrocardiogram signal processing device 100′ and/or the electrocardiogram management server 300 may differentially perform a function of storing an electrocardiogram signal and/or data for the electrocardiogram signal and a function of processing the electrocardiogram signal. The electrocardiogram signal processing device 100′ may divide the electrocardiogram signal into signal segments and cluster or group the signal segments. The electrocardiogram signal processing device 100′ may generate data from a processed result. The data for the processed result may include data for the result of dividing an electrocardiogram signal, data for the result of clustering or grouping signal segments, data for a noise section, and data for the analysis target section, and is not limited thereto, and may further include data obtained by processing the electrocardiogram signal.

The computing device 400 may include a processor, a communication unit, and a memory, access the electrocardiogram signal processing device 100′ through a network, and receive data generated by the electrocardiogram signal processing device 100′. The computing device 400 may download data stored in the electrocardiogram management server 400 and process the data through the electrocardiogram signal processing device 100′. The computing device 400 may access the electrocardiogram signal processing device 100′ through a network and receive result data of the electrocardiogram signal processed by the electrocardiogram signal processing device 100′. The computing device 400 may refer to a device that an analyst accesses. The computing device 400 may be a device of a user having access rights to the electrocardiogram management server 300 and/or the electrocardiogram signal processing device 100′. The computing device 400 may access the electrocardiogram management server 300 and/or the electrocardiogram signal processing device 100′ through a preset connection program to download an electrocardiogram signal, target object information, information on measurement, and/or data related to analysis, and may instruct the electrocardiogram management server 300 and/or the electrocardiogram signal processing device 100′ to classify a noise section included in the electrocardiogram signal and the analysis target section and generate an electrocardiogram signal from which the noise section is removed. The computing device 400 may store data for the electrocardiogram signal processed through the above process. The computing device 400 may modify a noise section of the data for an electrocardiogram signal as a non-noise section or a normal section thereof as the noise section. The computing device 400 may analyze data for an electrocardiogram signal to generate analysis data for each signal segment. The computing device 400 may acquire a comment from at least one of a medical staff, an analyst, and a patient. The computing device 400 may generate a report based on data for an electrocardiogram signal, analysis data for the electrocardiogram signal, comments, and/or so on. The computing device 400 may upload the data for an electrocardiogram signal, the analysis data, the comments, and/or the report to the electrocardiogram management server 300.

The electrocardiogram signal processing device 100′ may be connected to the electrocardiogram measurement device T1 through a communication network and receive a measured electrocardiogram signal. The electrocardiogram signal processing device 100′ may transmit an electrocardiogram signal measured by the electrocardiogram measurement device T1 to the electrocardiogram management server 300. The electrocardiogram signal processing device 100′ may receive an electrocardiogram signal from the electrocardiogram management server 300. The electrocardiogram signal processing device 100′ may request the electrocardiogram management server 400 to transmit an electrocardiogram signal to be processed. The electrocardiogram signal processing device 100′ may receive target object information of an electrocardiogram signal from the electrocardiogram management server 300. The electrocardiogram signal processing device 100′ may receive information on an electrocardiogram pattern of target object information from the electrocardiogram management server 300 through the target object information of an electrocardiogram signal. The electrocardiogram signal processing device 100′ may perform grouping or clustering to classify noise sections by using information on the electrocardiogram pattern. The electrocardiogram signal processing device 100′ may determine a reference signal segment or a reference value serving as a reference for a noise section by using information on the electrocardiogram pattern.

As described above, the electrocardiogram signal processing device 100′, the electrocardiogram management server 300, and the computing device 400 are described as separate devices but may be implemented in various combinations or configurations.

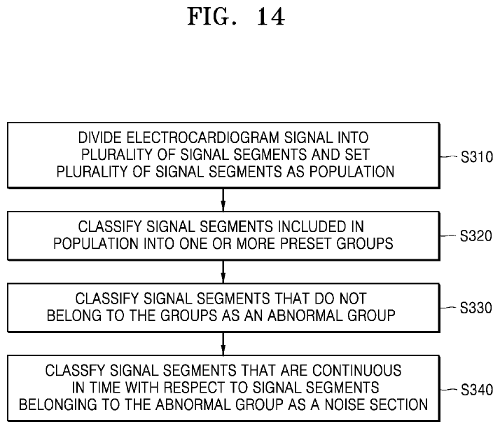

FIG. 14 is a flowchart of an electrocardiogram processing method according to embodiments.

As illustrated in FIG. 14, in step S310, the electrocardiogram signal processing device 100 may divide an electrocardiogram signal into a plurality of signal segments. As illustrated in FIG. 10, the electrocardiogram signal processing device 100 may divide an electrocardiogram signal into signal segments based on one signal pattern SSP. Here, the electrocardiogram signal may include an electrocardiogram measured for a preset period of time, for example, 48 hours, but is not limited thereto and may include electrocardiogram signals of various lengths. The electrocardiogram signal processing device 100 may set a plurality of divided signal segments as a total set. Here, the plurality of divided signal segments may each include a preset number of PQRST cycles and include one PQRST. Here, PQRST is for the sake of convenience, and it is natural that PQRST may be differentially processed as a heartbeat. The electrocardiogram signal processing device 100 may define a predetermined number of signal segments as one window. The electrocardiogram signal processing device 100 may process the signal in units of windows. The length of the windows can be changed.

The electrocardiogram signal processing device 100 may segment an electrocardiogram signal based on a signal pattern. For example, the electrocardiogram signal processing device 100 may divide an electrocardiogram signal into signal segments by dividing the electrocardiogram signal into repeating patterns. Here, the pattern of an electrocardiogram signal may be segmented based on at least one of peaks, P peaks, R peaks, and QRS time intervals of an electrocardiogram signal. The electrocardiogram signal processing device 100 may segment an electrocardiogram signal into patterns including points (time values and measurement values) of peaks. The electrocardiogram signal processing device 100 may segment an electrocardiogram signal into patterns of characteristics (time value and measurement value) of P peaks. The electrocardiogram signal processing device 100 may segment an electrocardiogram signal into patterns of characteristics (time value and measurement value) of R peaks. The electrocardiogram signal processing device 100 may segment an electrocardiogram signal based on the QRS time interval. Accordingly, it is possible to divide an electrocardiogram signal into signal segments having characteristics of patterns included in the measured electrocardiogram signal. In addition, it is possible to perform signal processing by considering a pattern included in each electrocardiogram signal. The electrocardiogram signal processing device 100 may normalize signal segments of an electrocardiogram signal. Accordingly, it is possible to perform classification according to a relative value rather than an absolute value. The electrocardiogram signal processing device 100 may segment an electrocardiogram signal into signal segments. For example, the signal segments may each include one PQRST or a preset number of PQRSTs.

In step S320, the electrocardiogram signal processing device 100 may classify signal segments included in a total set into one or more preset groups. The electrocardiogram signal processing device 100 may classify signal segments in units of windows. The electrocardiogram signal processing device 100 may classify all of the signal segments included in first window. Based on the information (e.g. group information) about the first window, the size of the second window after the first window may be determined. When the abnormal group is not detected in the first window, the length of the second window may be determined to be longer than the length of the first window. The length of the second window can be set to twice the length of the first window. When an abnormal group is detected in the first window, the length of the second window may be shorter than the length of the first window. Optionally, the electrocardiogram signal processing device 100 may normalize the signal segments and classify the signal segments into one or more groups. Each group may be classified as a result of grouping signal segments. The electrocardiogram signal processing device 100 may group signal segments of an electrocardiogram signal through clustering and classify the signal segments into one or more groups. For example, a clustering method may include K-means clustering, mean-shift clustering, density-based spatial clustering (DBSCAN: density-based spatial clustering of application with noise), and so on.

Grouping and/or clustering of electrocardiogram signals may be based on signal patterns of electrocardiogram signals. An electrocardiogram signal measured at one time may include repetition of similar patterns of signal segments. A section deviating from the similar patterns may correspond to noise. The electrocardiogram signal processing device 100 may analyze patterns of reference signal segments of an electrocardiogram signal and classify a section deviating from a similar pattern as a noise section. According to embodiments, the section deviating from the similar pattern may be determined as one or more reference signal segments or one or more reference values of complexity values.

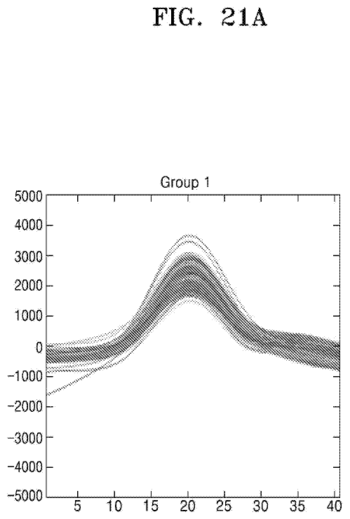

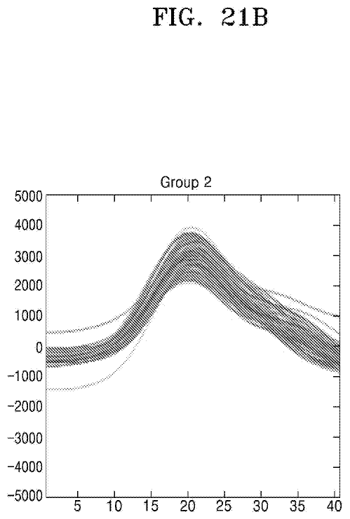

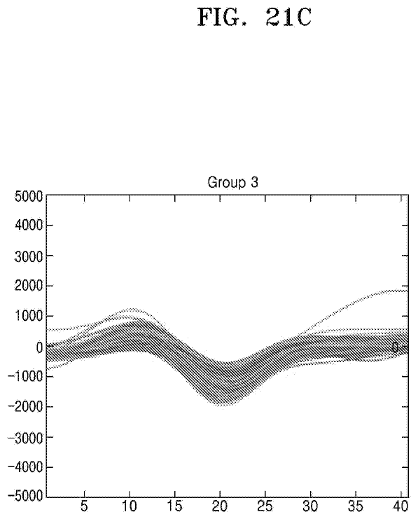

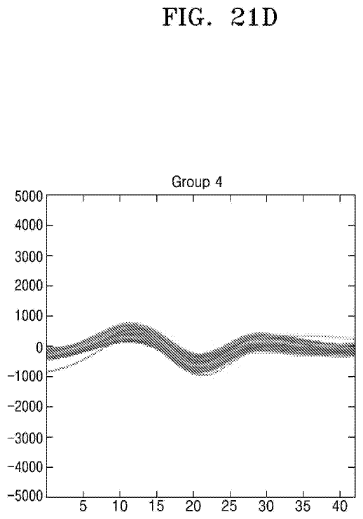

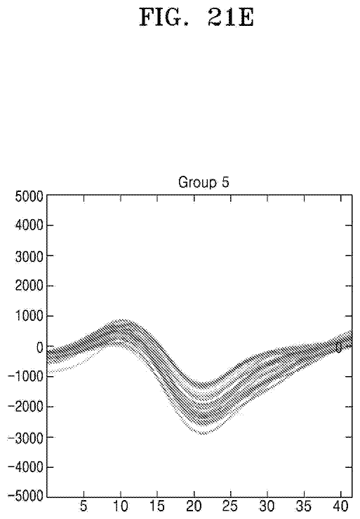

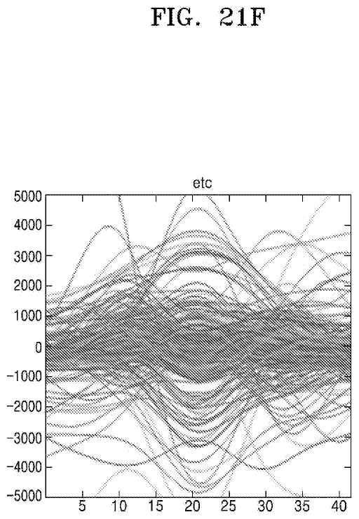

As illustrated in FIGS. 21A, 21B, 21C, 21D, and 21E, the electrocardiogram signal processing device 100 may classify signals into groups of Group 1, Group 2, Group 3, Group 4, And Group 5, and may classify signals that do not belong to the groups of Group 1, Group 2, Group 3, Group 4, and Group 5 as an abnormal group.

The electrocardiogram signal processing device 100 may classify Group 1 (see FIG. 21A) based on one reference signal segment or reference value belonging to group 1 (see FIG. 21A). The electrocardiogram signal processing device 100 may classify Group 2 (see FIG. 21B) based on one reference signal segment or reference value belonging to Group 2 (see FIG. 21B). The electrocardiogram signal processing device 100 may classify Group 3 (see FIG. 21C) based on one reference signal segment or reference value belonging to Group 3 (see FIG. 21C). The electrocardiogram signal processing device 100 may classify Group 4 (see FIG. 21D) based on one reference signal segment or reference value belonging to Group 4 (see FIG. 21D). The electrocardiogram signal processing device 100 may classify Group 5 (see FIG. 21E) based on one reference signal segment or reference value belonging to Group 5 (see FIG. 21E).

In step S330, the electrocardiogram signal processing device 100 may classify signal segments that do not belong to the groups as an abnormal group. Signal segments that do not belong to the groups are as illustrated in FIG. 21F as “etc.”.

In step S340, the electrocardiogram signal processing device 100 may classify signal segments that are continuous in time with respect to signal segments belonging to the abnormal group as a noise section. The electrocardiogram signal processing device 100 may determine the noise section in units of windows. The electrocardiogram signal processing device 100 may read all of the signal segments included in first window and determine whether or not the all of the signal segments include noise section. Based on the information (e.g. noise section) about the first window, the length of the second window after the first window may be determined. When the abnormal group is not detected in the first window, the length of the second window may be determined to be longer than the length of the first window. The length of the second window can be set to twice the length of the first window. When an abnormal group is detected in the first window, the length of the second window may be shorter than the length of the first window. Signal segments of the electrocardiogram signal that are not classified as noise may be set as an analysis target section. The electrocardiogram signal processing device 100 may set signal segments belonging to Group 1, Group 2, Group 3, Group 4, and Group 5 as the analysis target section, and set signals belonging to the abnormal group (see FIG. 21F) as a noise section. In another embodiment, the electrocardiogram signal processing device 100 may classify signal segments that satisfy a noise classification criterion among signal segments belonging to the abnormal group as a noise section. Here, the noise classification criterion may be set as etc. signal segments generated continuously in time or three signal segments generated continuously in time. The noise classification criterion may be determined by the noise decision model.

The electrocardiogram signal processing device 100 may determine whether signal segments classified as the abnormal group are temporally adjacent signal segments. The electrocardiogram signal processing device 100 may insert group information of each of the signal segments into an electrocardiogram signal as a result of clustering. Data of an electrocardiogram signal into which group information is inserted may be as illustrated in FIGS. 22A and 22B. The electrocardiogram signal processing device 100 may generate data by listing pieces of group information of signal segments of an electrocardiogram signal in a time dimension. The electrocardiogram signal processing device 100 may scan the pieces of group information of the signal segments in this data to detect a first signal segment classified as the abnormal group and determine whether a second signal segment subsequent the first signal segment corresponds to the abnormal group. When both the first signal segment and the second signal segment belong to the abnormal group, the first signal segment and the second signal segment may be classified as a noise section. When the second signal segment corresponds to the one or more group, the first signal segment may be classified as an analysis target section.

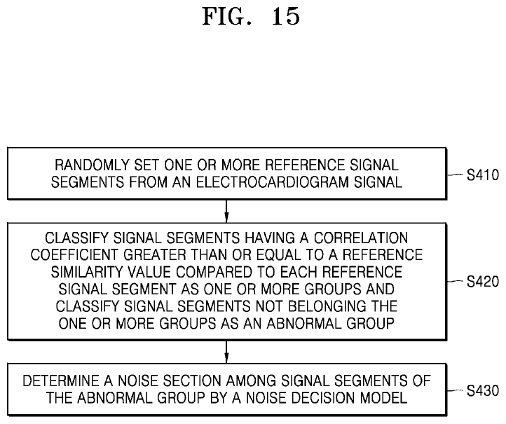

FIG. 15 is a flowchart of a method of setting, by the electrocardiogram signal processing device 100, an abnormal group by using a reference segment, according to embodiments.

Accordingly, the electrocardiogram signal may be measured by a single channel measuring device. The electrocardiogram signal may have different characteristics depending on an attachment position, a heart state of an object, or a heart movement of the object. The electrocardiogram signals measured from the same object may have different characteristics depending on the measurement point or the measurement time. The electrocardiogram signal measured by a single-channel measuring device individually includes signal patterns that change each time measurement is performed. In order to determine a noise section in the measured electrocardiogram signal, a process of determining a reference value or reference segment for each electrocardiogram signal may be required.

In step S410, the electrocardiogram signal processing device 100 may randomly set one or more reference signal segments from an electrocardiogram signal. For example, the reference signal segment may be set based on the frequency of occurrence in an electrocardiogram signal. The reference signal segment may vary by measurement location or object. When the electrocardiogram signal processing device 100 calculates the frequency of occurrence based on a pattern of each signal segment using deep learning model or machine learning model trained, one or more reference signal segments may be set in descending order of the frequency of occurrence. Optionally, the reference signal segment may be determined using the electrocardiogram classification model. The electrocardiogram classification model may be a model obtained by learning data about feature points, such as R-R intervals and QRS patterns, of frequently generated electrocardiogram signals by taking a plurality of electrocardiogram signals as inputs. The electrocardiogram classification model may classify electrocardiogram signals into one or more groups based on the learned data.

In step S420, the electrocardiogram signal processing device 100 may classify signal segments having a correlation coefficient greater than or equal to a reference similarity value compared to each reference signal segment as one or more groups while scanning an electrocardiogram signal. The electrocardiogram signal processing device 100 may classify signal segments in units of windows. The electrocardiogram signal processing device 100 may classify all of the signal segments included in first window. When the abnormal group is not detected in the first window, the length of the second window may be determined to be longer than the length of the first window. The length of the second window can be set to twice the length of the first window. The electrocardiogram signal processing device 100 may classify signal segments not belonging the one or more groups as an abnormal group. The electrocardiogram signal processing device 100 may calculate a correlation coefficient obtained by comparing signal segments with a reference signal segment as a degree of similarity. The reference similarity value may be set to a correlation coefficient of 0.9. The reference similarity value is not limited thereto and may be set to various values. The electrocardiogram signal processing device 100 may classify signal segments similar to respective reference signal segments into the one or more groups.

Optionally, the electrocardiogram signal processing apparatus 100 may classify the signal segments into one or more groups using the electrocardiogram classification model. The electrocardiogram classification model may classify signal segments using feature values such as similarity, complexity, or pattern similarity.

In step S430, the electrocardiogram signal processing device 100 may determine the noise section among signal segments among the abnormal group by the noise decision model. The electrocardiogram signal processing device 100 may generate the analysis target section associated with the electrocardiogram signal excluding the noise section. The electrocardiogram signal processing device 100 may determine the noise section in units of windows. The electrocardiogram signal processing device 100 may read all of the signal segments included in first window and determine whether or not the all of the signal segments include noise section. Based on the information (e.g. noise section) about the first window, the length of the second window after the first window may be determined. When the noise section is not detected in the first window, the length of the second window may be determined to be longer than the length of the first window. The length of the second window can be set to twice the length of the first window. When the noise section is detected in the first window, the length of the second window may be shorter than the length of the first window.

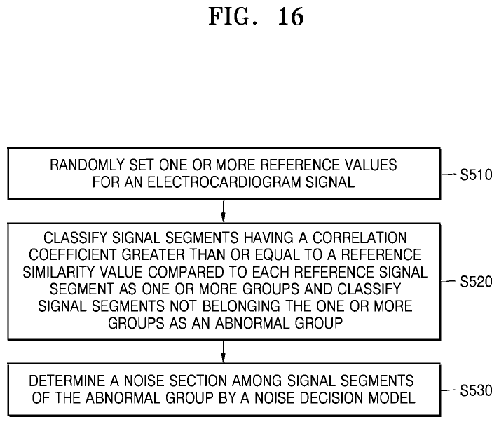

FIG. 16 is a flowchart of a method of classifying, by an electrocardiogram signal processing device, an abnormal group by using a reference value, according to embodiments.

In step S510, the electrocardiogram signal processing device 100 may set one or more reference values for an electrocardiogram signal. The one or more reference value for an electrocardiogram signal may be set based on the frequency of occurrence. The one or more reference value may be determined using the electrocardiogram classification model trained by deep learning or machine learning. In another embodiment, the electrocardiogram signal processing device 100 may set the reference values based on complexity values of signal segments. The electrocardiogram signal processing device 100 may cluster or group signal segments of an electrocardiogram signal based on complexity value, time interval, or peak value and set a reference signal segment. The electrocardiogram signal processing device 100 may randomly select 50 signal segments from an electrocardiogram signal and set at least one of complexity values, time interval values, and peak values for the selected signal segments as reference values for the respective groups. For example, the complexity value may be determined in the frequency dimension. A signal segment that does not have a regular signal pattern may have a higher complexity value than the regular signal pattern. The frequency of a signal segment that does not have the regular signal pattern can be calculated higher than the regular signal pattern.

In step S520, the electrocardiogram signal processing device 100 may sequentially read an electrocardiogram signal, classify signal segments satisfying respective reference values as respective groups, and classify signal segments, which do not belong to the respective groups, as the abnormal group. Signal segments satisfying a condition set based on a reference value may be set as one group. For example, signal segments having a value exceeding a first reference value may be set as one group. Signal segments having a value that is less than or equal to a second reference value may be set as one group. Signal segments having values in a third reference range may be set as one group. The electrocardiogram signal processing device 100 may classify signal segments in units of windows. The electrocardiogram signal processing device 100 may classify all of the signal segments included in first window. When the abnormal group is not detected in the first window, the length of the second window may be determined to be longer than the length of the first window.

Here, the reference value may be set to a preset value or may be set to a preset range. The reference value may be set to at least one of a complexity value, a peak value, and a time interval value of a signal. The peak value may be the largest value within a signal segment. The time interval value may be a time cycle value of the signal segment. The reference value is not limited thereto, and various values may be used as the reference value. The reference value may vary by a measurement location or an object.

In step S530, the electrocardiogram signal processing device 100 may determine the noise section among signal segments among the abnormal group by the noise decision model. The electrocardiogram signal processing device 100 may generate the analysis target section associated with the electrocardiogram signal excluding the noise section. The electrocardiogram signal processing device 100 may determine the noise section in units of windows. The electrocardiogram signal processing device 100 may read all of the signal segments included in first window and determine whether or not the all of the signal segments include noise section. Based on the information (e.g. noise section) about the first window, the length of the second window after the first window may be determined. When the noise section is not detected in the first window, the length of the second window may be determined to be longer than the length of the first window. The length of the second window can be set to twice the length of the first window. When the noise section is detected in the first window, the length of the second window may be shorter than the length of the first window.

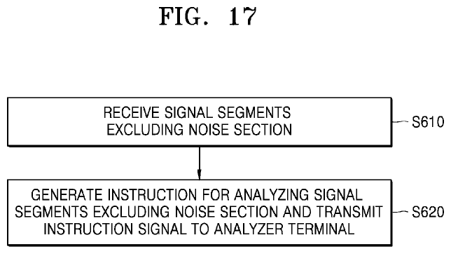

FIG. 17 is a flowchart of a method of processing signal segments of an analysis target section, according to embodiments.

In step S610, an electronic device may receive data for signal segments in an analysis target section excluding a noise section from the electrocardiogram signal processing device 100 or the electrocardiogram management server 300. The electronic device may include a processor, a communication unit, and a memory. The electronic device may receive signal segments excluding the noise section. The analyst or medical staff may load the data of the analysis target section to analyze the analysis target section. An analyst or medical staff may input data on whether or not the subject has a heart disease, a heart risk state, and the like by analyzing an analysis target section provided by the electronic device.

In step S620, the electronic device may generate an instruction signal for analyzing signal segments excluding a noise section and transmit the instruction signal to an analysis device. Here, the electronic device may be at least one of 200 in FIGS. 12 and 200, 300, and 400 in FIG. 13.

The electronic device may generate analysis data for the electrocardiogram signal by inputting the data of the analysis target section to an electrocardiogram analysis algorithm. The electrocardiogram analysis algorithm is be learned by deep learning or machine learning. By classifying a noise section of an electrocardiogram signal, a reference signal segment or a reference value may be determined within the electrocardiogram signal as described above, but the present disclosure is not limited thereto, and the reference signal segment and/or the reference value may be determined by using an electrocardiogram signal previously measured for a target object of the electrocardiogram signal. In another embodiment, classifying a noise section of an electrocardiogram signal is not limited to a target object, and a reference signal segment and/or a reference value may be determined by using patterns of noise sections classified in the measured electrocardiogram signals.

According to embodiments, each group may be based on a classification criterion for an electrocardiogram signal. When designed to be classified as one of a normal beat (N), a supraventricular ectopy beat (S), and a ventricular ectopy beat (V), a first group may be classified as a reference signal segment corresponding to the normal beat (N). A second group may be classified as a reference signal segment corresponding to the supraventricular ectopy beats (S). A third group may be classified as a reference signal segment corresponding to the ventricular ectopy beat (V).

Additionally, according to embodiments, when the normal beat is classified as one of R-R pause, bradycardia, N-N delay, heart block, and atrial fibrillation, reference signal segments corresponding to each thereof may be determined, and signal segments may be classified by the respective reference signal segments. In this case, by determining which reference signal segment each signal segment is similar to, which classification criterion the signal segment corresponds to may be determined. In one operation, a signal segment may be classified as one of the one or more groups.

Herein, the electronic device may be one of 200 in FIGS. 12 and 200, 300, and 400 in FIG. 13.

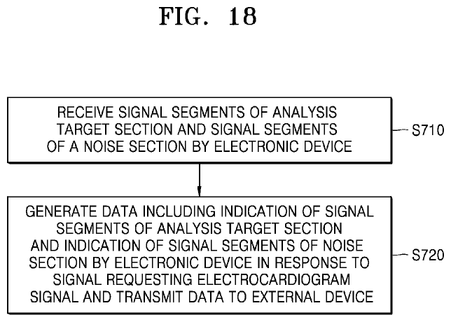

FIG. 18 is a flowchart of a method of processing an electrocardiogram signal, according to embodiments.

In step S710, an electronic device may receive signal segments of an analysis target section and signal segments of a noise section.

In step S720, the electronic device may generate data including an indication of signal segments of the analysis target section and an indication of signal segments of the noise section in response to a signal requesting an electrocardiogram signal, and transmits the data to an external device. Here, the indications may include a tag, event information, group information, time information, or object information added to the electrocardiogram signal and display information on an event. The event information is information about discomfort felt by an object in relation to a heart, and may be input by the object. The event information may include a time value at which the object felt discomfort and intensity of the discomfort. The group information may be group information to which each signal segment belongs. The time information may include a time value for the analysis target section and a time value for the noise section. The object information may include the age, gender, occupation, medical history, and physical information of the object.

Herein, the electronic device may be one of 200 in FIGS. 12 and 200, 300, and 400 in FIG. 13.

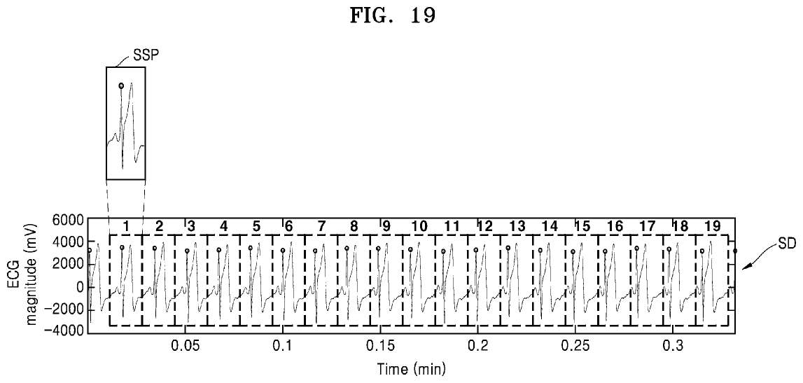

FIG. 19 is an example diagram illustrating that an electrocardiogram signal is divided into signal segments, according to embodiments.

An electrocardiogram signal SD may be segmented into signal segments 1, 2, 3, 4, 5, 6, 7, 8, 9, 10, 11, 12, 13, 14, 15, 16, 17, 18, and 19 based on patterns SSP of the electrocardiogram signal SD. As illustrated in FIG. 19, the patterns SSP of the electrocardiogram signal may each include one PQRST. The electrocardiogram signal processing device 100 may group or cluster the segmented signal segments 1, 2, 3, 4, 5, 6, 7, 8, 9, 10, 11, 12, 13, 14, 15, 16, 17, 18, and 19 into similar segments.

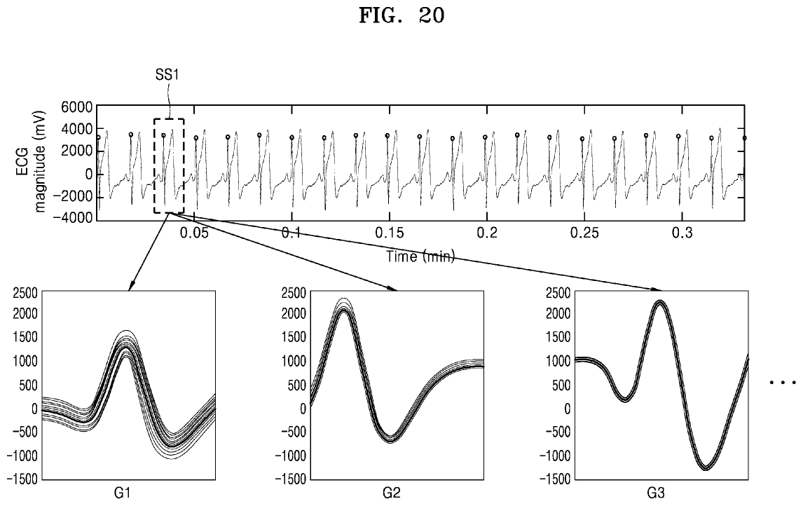

FIG. 20 is an example diagram illustrating a process of classifying electrocardiogram signals into respective groups, according to embodiments.

As illustrated in FIG. 20, according to embodiments, an electrocardiogram signal may be scanned in units of signal segments, and which group the signal segment belongs to may be detected. In FIG. 20, arrows indicate that SS1 is connected to G1, G2, and G3. While reading an electrocardiogram signal in units of SS1, whether SS1 belongs to G1, G2, or G3 is determined, and SS1 may be classified as one of G1, G2, or G3.

FIG. 21A is an example diagram illustrating signal segments belonging to a first group classified according to embodiments.

The electrocardiogram signal processing device 100 may classify Group 1 (see FIG. 21A) based on one reference signal segment or a reference value belonging to Group 1 (see FIG. 21A).

FIG. 21B is an example diagram illustrating signal segments belonging to a second group classified according to embodiments.

The electrocardiogram signal processing device 100 may classify Group 2 (see FIG. 21B) based on one reference signal segment or a reference value belonging to Group 2 (see FIG. 21B).

FIG. 21C is an example diagram illustrating signal segments belonging to a third group classified according to embodiments.

The electrocardiogram signal processing device 100 may classify Group 3 (see FIG. 21C) based on one reference signal segment or a reference value belonging to Group 3 (see FIG. 21C).

FIG. 21D is an example diagram illustrating signal segments belonging to a fourth group classified according to embodiments.

The electrocardiogram signal processing device 100 may classify Group 4 (see FIG. 21D) based on one reference signal segment or a reference value belonging to Group 4 (see FIG. 21D).

FIG. 21E is an example diagram illustrating signal segments belonging to a fifth group classified according to embodiments.

The electrocardiogram signal processing device 100 may classify Group 5 (see FIG. 21E) based on one reference signal segment or a reference value belonging to Group 5 (see FIG. 21E).

FIG. 21F is an example diagram illustrating signal segments belonging to the abnormal group classified according to embodiments.

The electrocardiogram signal processing device 100 may classify Group 5 (see FIG. 21E) based on one reference signal segment or a reference value belonging to Group 5 (see FIG. 21E).

According to embodiments, electrocardiogram signals may be classified into five groups illustrated in FIGS. 21A, 21B, 21C, 21D, and 21E and may be classified as the abnormal group illustrated in FIG. 21F. Signal segments classified as the abnormal group may be set as a noise section.

According to the embodiments, it is possible to determine which group each signal segment belongs to and which noise section each signal segment corresponds to by performing one operation on signal segments of one cycle of an electrocardiogram signal. This enables the analysis of each signal segment to be completed in one operation regardless of the number of classification criteria.

FIG. 22A is an example diagram of a section other than a noise section processed according to embodiments of the present disclosure.

The electrocardiogram signal processing device 100 may cluster an electrocardiogram signal, and as a result of the clustering, when signal segments classified as the abnormal group are continuous in time, the corresponding signal segments may be classified as a noise section. As illustrated in FIG. 22A, an electrocardiogram signal may be illustrated as a graph of time and amplitude. According to embodiments, data may be generated in which points P11, P12, P13, P14, P15, and P16 corresponding to peaks of signal segments are marked to an electrocardiogram signal. As a result of clustering by the electrocardiogram signal processing device 100, the point P11, P12, P14, P15, and P16 correspond to peaks of signal segments clustered as a preset group. The point P13 corresponds to the peak of the signal segment clustered as the abnormal group. The point P13 of an electrocardiogram signal which is classified as the abnormal group is not continuous in time. According to embodiments, the electrocardiogram signal processing device 100 may classify the signal segment including the point P13 classified as the abnormal group as a section other than the noise section. The signal segment including the point P13 may be classified as a normal section.

FIG. 22B is an example diagram of a noise section processed according to embodiments.

As illustrated in FIG. 22B, an electrocardiogram signal may include points P21, P22, P23, P24, P25, P26, P27, and P28 corresponding to respective signal segments. According to embodiments, the points P21, P22, P23, and P28 among the points P21, P22, P23, P24, P25, P26, P27, and P28 are classified as a preset group, and the points P24, P25, P26, and P27 are classified as the abnormal group. Signal segments classified as the abnormal group, such as the points P24, P25, P26, and P27, may be generated continuously in time, and signal segments corresponding to the points P24, P25, P26, and P27 may be classified as a noise section. According to the embodiments, time of windows can be dynamically adjustable. According to a conventional art, an electrocardiogram signal processing device uses a given time of windows including signal segments, all of the signal segments in a time of window that includes one or more noise segments may be classified as a noise sections and excluded in a further classification or a further analysis. The present disclosure may be applied to adjust the time of window, so that loss of non-noise segments (normal signal segment) can be minimized. According to the embodiments, analysis by excluding normal signal segment can be minimized. Four signal segments classified as the abnormal group may be continuous in time, and a plurality of temporally continuous signal segments may be classified as a noise section.

The device described above may be implemented with hardware components, software components, and/or a combination of hardware components and software components. For example, the device and components described in the embodiments may be implemented with one or more general purpose computers or special purpose computers, such as a processor, a controller, an arithmetic logic unit (ALU), a digital signal processor, a microcomputer, a field programmable gate array (FPGA), a programmable logic unit (PLU), a microprocessor, or any other device capable of executing and responding to instructions. A processing device may execute an operating system (OS) and one or more software applications executed on the operating system. In addition, the processing device may also access, store, operate, process, and generate data in response to execution of software. For the sake of convenient understanding, one processing device may be used, but those skilled in the art will understand that the processing device may include a plurality of processing elements and/or multiple types of processing elements. For example, the processing device may include a plurality of processors or one processor and one controller. In addition, other processing configurations such as a parallel processor are possible.

The software may include a computer program, code, instructions, or a combination thereof, may operate the processing device as desired, or may independently or collectively instruct the processing device. Software and/or data may be interpreted by a processing device or may be permanently or temporarily embodied for any type of machine, component, physical device, virtual equipment, computer storage medium or device, or signal wave being transmitted to provide instructions or data to a processing device. Software may also be distributed to computer systems connected to each other via a network to be stored or executed in a distributed manner. Software and data may be stored in one or more computer-readable recording media.

The method according to the embodiment may be recorded in a computer-readable recording medium in the form of program instructions that may be executed through various computer systems. The computer-readable recording medium may include program instructions, data files, data structures, and so on alone or in combination. Program instructions recorded on the computer-readable recording medium may also be specially designed and configured for the embodiment or may also be known and usable to those skilled in computer software. Computer-readable recording media include, for example, magnetic media such as a hard disk, a floppy disk, and magnetic tape, optical media such as a compact disc read-only mem (CD-ROM) and a digital video disk (DVD), magneto-optical media such as floptical disks, and a hardware device which is specially configured to store and execute program instructions, such as read only memory (ROM), random access memory (RAM), or flash memory. Program instructions include, for example, not only machine language code such as code generated by a compiler, but also high-level language code that may be executed by a computer by using an interpreter or so on. The hardware device may be configured to operate as one or more software modules to perform an operation of the embodiment, and vice versa.

According to the method of processing an electrocardiogram signal, according to the present disclosure, an abnormal electrocardiogram signal may be accurately determined by determining whether or not the electrocardiogram signal is abnormal through two steps.

According to embodiments, a noise section may be detected from an electrocardiogram signal.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A non-transitory computer-readable storage medium storing instruction that, when executed by a processor, causes the processor to perform operations comprising:
acquiring an electrocardiogram signal of an object;
generating first signal segmentations by dividing the electrocardiogram signal according to a division rule;
classifying the first signal segments into one or more preset groups using an electrocardiogram classification model and classifying second signal segments that do not belong to the one or more preset groups into an abnormal group;
determining a noise section associated with the electrocardiogram signal among the second signal segments using a noise decision model;
generating an analysis target section associated with the electrocardiogram signal excluding the noise section; and
transmitting the analysis target section to an external device to analyze information related to a heart of the object.

2. The non-transitory computer-readable storage medium of claim 1, wherein the electrocardiogram classification model is learned using signal segments of electrocardiogram signals measured in a plurality of objects as input using a deep learning method or a machine learning model to classify the first signal segments into the one or more preset groups and classifying the second signal segments into the abnormal group using a degree of similarity.

3. The non-transitory computer-readable storage medium of claim 1, wherein the electrocardiogram classification model is learned using signal segments of electrocardiogram signals measured in a plurality of objects as input using a deep learning method or a machine learning model to classify the first signal segment into the one or more preset groups and classifying the second signal segments into the abnormal group using a degree of complexity.

4. The non-transitory computer-readable storage medium of claim 1, wherein the division rule is to divide into signal segments based on time intervals or QRS time intervals of peaks of the electrocardiogram signal.

5. The non-transitory computer-readable storage medium of claim 1, wherein the division rule is adjustable based on whether or not a noise section is detected in a previous time-of-window.

6. The non-transitory computer-readable storage medium of claim 1, wherein the noise decision model is to determine signal segments continuously generated more than a reference number of times as the noise section among the second signal segments.

7. The non-transitory computer-readable storage medium of claim 5, wherein a reference number is a value determined using a deep learning model or a machine learning model learned with a plurality of electrocardiogram signals of a plurality of objects as input.

8. The non-transitory computer-readable storage medium of claim 6, wherein a deep learning model or machine learning model is learned with first signal segments including a noise section and second signal segments not including a noise section.

9. The non-transitory computer-readable storage medium of claim 1, wherein the electrocardiogram classification model includes a plurality of models and is operated by selecting a model according to a measurement position.

10. A method of processing an electrocardiogram signal by an electrocardiogram signal processing device including at least one processor, the method comprising:
acquiring an electrocardiogram signal of an object;
generating first signal segmentations by dividing the electrocardiogram signal according to a division rule;
classifying the first signal segments into one or more preset groups using an electrocardiogram classification model and classifying second signal segments that do not belong to the one or more preset groups into an abnormal group;
determining a noise section associated with the electrocardiogram signal among the second signal segments using a noise decision model;
generating an analysis target section associated with the electrocardiogram signal excluding the noise section; and
transmitting the analysis target section to an external device to analyze information related to a heart of the object.

11. The method of claim 10, wherein the electrocardiogram classification model is learned using signal segments of electrocardiogram signals measured in a plurality of objects as input using a deep learning method or a machine learning model to classify the first signal segments into the one or more preset groups and classifying the second signal segments into the abnormal group using a degree of similarity.

12. The method of claim 10, wherein the electrocardiogram classification model is learned using signal segments of electrocardiogram signals measured in a plurality of objects as input using a deep learning method or a machine learning model to classify the first signal segments into the one or more preset groups and classifying the second signal segments into the abnormal group using a degree of complexity.

13. The method of claim 10, wherein the division rule is to divide into signal segments based on time intervals or QRS time intervals of peaks of the electrocardiogram signal.

14. The method of claim 10, wherein the noise decision model is to determine signal segments continuously generated more than a reference number of times as the noise section among the second signal segments.

15. The method of claim 14, wherein the reference number is a value determined using a deep learning model or a machine learning model learned with a plurality of electrocardiogram signals of a plurality of objects as input.

16. The method of claim 15, wherein the deep learning model or the machine learning model is learned with signal segments including a noise section and signal segments not including a noise section.

17. The method of claim 10, wherein the electrocardiogram classification model includes a plurality of models and is operated by selecting a model according to a measurement position.
